(19) United States
(12) Patent Application Publication
(10) Pub. No.: US 2026/0306481 A1
(43) Pub. Date: Oct. 8, 2026

(54) RAPID INDUCTION WITH MONTHLY INJECTABLE BUPRENORPHINE EXTENDED-RELEASE

(71) Applicant: Indivior UK Limited, Hull (GB)

(72) Inventors: Christian A. HEIDBREDER, North Chesterfield, VA (US); Frank GRAY, London (GB); Celine M. LAFFONT, North Chesterfield, VA (US); Robert L. DOBBINS, North Chesterfield, VA (US)

(21) Appl. No.: 19/704,160

(22) Filed: Jun. 10, 2026

Related U.S. Application Data
(63) Continuation of application No. 19/640,311, filed on Apr. 6, 2026.
(60) Provisional application No. 63/783,552, filed on Apr. 4, 2025.

(51) Int. Cl. A61K 31/485; A61K 9/00; A61P 25/36

(52) U.S. Cl. CPC A61K 31/485; A61K 9/0019; A61K 9/006; A61P 25/36

(57) ABSTRACT

The disclosure provides methods of treating opioid use disorder in a human by rapid induction with monthly injectable extended release buprenorphine, and to rapid induction dosage regimens for treating opioid use disorder. The methods provide increased treatment retention compared to standard of care methods.

### CROSS-REFERENCE TO RELATED APPLICATION

This application is a continuation of U.S. patent application Ser. No. 19/640,311, filed Apr. 6, 2026, which claims priority to U.S. Provisional Patent Application No. 63/783,552, filed Apr. 4, 2025, the contents of which are incorporated herein in their entireties.

### TECHNICAL FIELD

The disclosure relates to methods of treating opioid use disorder in a human by rapid induction with monthly injectable buprenorphine extended-release, and to rapid induction dosage regimens for treating opioid use disorder.

### BACKGROUND

Opioids are powerful medications and drugs which are prescribed following injury or surgery to relieve moderate-to-severe pain to enable activity, or for disease conditions including cancer. Opioids include substances such as morphine, fentanyl, codeine, hydrocodone, oxycodone, oxymorphone, hydromorphone, tapentadol, and methadone. Due to the widespread availability and variety of prescription opioid products, the number of opioid prescriptions is significantly high (153 million in 2019) in the United States alone.

The inappropriate use of opioids often causes serious problems such as addiction, abuse, overdose, and dependence on opioids, known as opioid use disorder (“OUD”). OUD is a chronic, relapsing disease associated with an elevated risk of mortality and morbidity that has been described as one of the most challenging forms of addictions. According to the Diagnostic and Statistical Manual for Mental Disorders, 5^{th} Edition (“DSM-5”), OUD is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. OUDs affect over 16 million people worldwide, over 2.1 million in the United States among them, and cause over 120,000 deaths worldwide annually attributed to opioids. The U.S. Department of Health and Human Services reports that overdose deaths involving opioids increased 519.38% from 1999 to 2019. In addition to these serious issues, the use of opioids can cause a number of side effects including increased opioid tolerance, symptoms of withdrawal when discontinued, increased sensitivity to pain, nausea, and/or depression.

Opioid receptors are located in both the central nervous system (“CNS”) and the periphery. In the CNS, they are found in high concentrations in the limbic system and the spinal cord. The natural ligands for the opioid receptors are a group of neuropeptides known as endorphins. Opioid analgesics mimic the action of these natural ligands but have a more prolonged action as they are not subject to rapid local metabolism. Three major opioid receptor subclasses have been identified: μ-, κ-, and δ-.

Buprenorphine is a semisynthetic derivative of thebaine and is a mixed partial agonist opioid receptor modulator with the chemical formula C₂₉H₄₁NO₄, and chemical name 21-cyclopropyl-7α-[(S)-1-hydroxy-1,2,2-trimethylpropyl]-6,14-endo-ethano-6,7,8, 14-tetrahydrooropavine. Buprenorphine is a partial opioid agonist at the μ-opioid receptor with antagonist properties at the K-receptor, and has been used for medication-assisted treatment of OUD. In contrast to a full agonist, buprenorphine at the μ-receptor has less maximal euphoric effect, and a ceiling on its respiratory depressant effects. By binding to μ-opioid receptors in the brain, buprenorphine reduces craving for opioids and opiate withdrawal symptoms, minimizing the need of opioid-dependent patients to use illicit opiate drugs.

Buprenorphine is the active ingredient in immediate-release formulations such as transmucosal (“TM”) formulations like SUBOXONE®, and long-acting injectable formulations such as SUBLOCADE®. SUBOXONE® is a sublingual film that includes buprenorphine hydrochloride and naloxone hydrochloride in dosages of 2 mg/0.5 mg, 4 mg/1 mg, 8 mg/2 mg, or 12 mg/3 mg (as buprenorphine/naloxone). SUBLOCADE®, which comes in dosages of 100 mg or 300 mg buprenorphine free base, is the first monthly, extended-release buprenorphine injection (“BUP-XR”) approved in the U.S. for treatment of moderate-to-severe OUD. SUBLOCADE® was designed for dosing regimens selected to ensure patients are exposed to safe and therapeutic levels throughout the month, with no drop in buprenorphine plasma concentrations below target (generally about 2 ng/mL or higher) that would trigger re-emergence of opioid withdrawal, craving, or potential relapse to opioid use.

Buprenorphine induction is the first step in BUP-XR treatment. The goal of induction is to reach a therapeutic BUP concentration as soon as possible to manage OUD without inducing precipitated withdrawal that could jeopardize continued engagement (retention) in BUP-XR treatment. This is because precipitated withdrawal, which occurs upon cessation or significant decrease in opioid receptor activity after weeks or months of agonism by opioid antagonists such as fentanyl, leads to opioid withdrawal symptoms (“OWS”) such as chills, body aches, emesis, diarrhea, dysphoria, irritability, and anxiety. Fear of OWS and its undertreatment has been reported as a reason for delaying or discontinuing OUD treatment.

Currently, standard of care induction (“SoC”) methods of treating OUD include a stepwise dosing approach for administration of TM buprenorphine, which is done for purposes of patient safety and tolerance. On day 1, TM buprenorphine is typically given at an initial dose of 2 to 4 mg, with upwards titration in 2 to 4 mg increments up to a maximal daily dose of 8 to 16 mg. On day 2, TM buprenorphine dose is adjusted based on patient's evaluation. Typically, a single daily dose of 12 or 16 mg is administered. Treatment with BUP-XR then begins with administration of a first dose (e.g. via sub-cutaneous injection) after a minimum of seven (7) and up to fourteen (14) days of induction and dose stabilization with TM buprenorphine, followed by once-monthly administration of BUP-XR until either the treatment is complete or the patient discontinues treatment.

For patients who successfully initiate onto and remain in BUP-XR treatment, for example with monthly sub-cutaneous injections of SUBLOCADE®, a significant improvement in opioid abstinence, treatment retention, and health-related quality of life has been observed. However, it has been challenging to successfully initiate patients into, as well as to retain patients in, such treatment, especially in high-risk populations. For example, patients who use intravenous drugs, very high doses of opioids, or highly potent synthetic opioids have an increased risk of treatment failure and opioid overdose with SoC methods. It is understood that this is due, at least in part, to increased OWS severity in such patients, for example in patients who use fentanyl. In addition, because patients who use fentanyl react differently than non-fentanyl-using patients, successful induction in fentanyl patients is particularly challenging. Therefore, dosing regimens and treatment methods that reduce the risk of treatment failure compared to current dosage regimens and treatment methods, particularly in high-risk populations, are needed.

Surprisingly, it has been found that the rapid induction (“RI”) methods described herein have improved success in patient retention in treatment with BUP-XR. Thus, the present disclosure provides RI dosing regimens for buprenorphine, as well as dosing regimens and treatment methods that offer, among other benefits, optimal ways to reach and maintain buprenorphine dosages and target therapeutic plasma levels for the treatment of opioid dependence in patients, particularly in high-risk opioid users including patients who use fentanyl, compared to SI dosing regimens.

### SUMMARY

The disclosure relates to methods of treating OUD by RI with injectable BUP-XR, for example SUBLOCADE®, methods of increasing patient retention in OUD treatment with BUP-XR, methods of increasing patient retention in patients experiencing Precipitated Opioid Withdrawal (“POW”), methods of rapid buprenorphine induction, methods of improving Clinical Opiate Withdrawal Scores (“COWS”) during induction, and methods of achieving target buprenorphine plasma concentration. RI dosing regimens for treating OUD are also disclosed.

In some embodiments, the disclosure relates to methods of treating OUD in a human patient in need thereof and/or rapid buprenorphine induction in a human patient in need thereof, the methods comprising administering TM buprenorphine hydrochloride to the human patient and, within no more than about 24 hours, such as no more than about 12 hours, administering a first dose of BUP-XR to the human patient. The methods may further comprise administering one or more additional doses of BUP-XR. For example, the methods may comprise administering a second dose of BUP-XR to the human patient within about 2 to about 31 days, for example about one week, after the first dose, and may further optionally comprise administering a third dose of BUP-XR to the human patient about one month, for example about 28 to 31 days, after the second dose.

In further embodiments, the disclosure relates to methods of improving or increasing patient retention, including in patients experiencing POW, in treatment of OUD with BUP-XR, the methods comprising administering TM buprenorphine hydrochloride to the patient; within no more than about 24 hours, such as no more than about 12 hours, no more than about 6 hours, or no more than about 3 hours, administering a first dose of BUP-XR to the patient; within about 2 to about 31 days, for example about one week, administering a second dose of BUP-XR to the patient. The methods may further optionally comprise administering a third dose of BUP-XR to the patient about one month, for example about 28 to 31 days, after the second dose. Patient retention after the second dose and/or after the third dose may be improved compared to SI.

In further embodiments still, the disclosure relates to methods of achieving target buprenorphine plasma concentration in a human, the methods comprising administering TM buprenorphine hydrochloride to the human; within no more than about 24 hours, such as no more than about 12 hours, no more than about 6 hours, or no more than about 3 hours, administering a first dose of BUP-XR to the human; within about 2 to about 31 days, for example about one week, administering a second dose of BUP-XR to the human. Although the methods achieve the same or substantially the same target buprenorphine plasma concentration as SoC and SI induction dosing regimens, the amount of time needed to do so is surprisingly significantly less. Using the disclosed RI induction dosing regimens, target plasma concentrations are generally achieved in about one month or less after day 1, for example about 2 weeks or less, such as about one week after day 1. This reduction in time to target plasma concentration advantageously leads to higher initiation and retention success rates, particularly among high-risk opioid users.

Rapid induction of buprenorphine and dosing regimens for RI are also disclosed, by administering TM buprenorphine hydrochloride to the human and, within no more than about 24 hours, such as no more than about 12 hours, no more than about 6 hours, or no more than about 3 hours, administering a first dose of BUP-XR to the human, and within about 2 to about 31 days, for example about one week, administering a second dose of BUP-XR to the human.

Methods of improving COWS during induction, particularly for fentanyl-positive patients, are also disclosed, for example by administering TM buprenorphine hydrochloride to the human and, within no more than about 24 hours, such as no more than about 12 hours, no more than about 6 hours, or no more than about 3 hours, administering a first dose of BUP-XR to the human.

In various aspects of embodiments of the disclosure, TM buprenorphine hydrochloride may be administered at a dose of about 2 mg, about 4 mg, about 6 mg, 8 mg, about 10 mg, about 12 mg, about 14 mg, or about 16 mg. In individuals who are not already receiving TM buprenorphine hydrochloride, typically a dose ranging from about 2 mg to about 4 mg, such as 2 mg or 4 mg, preferably about 4 mg, is used. Administration of TM buprenorphine may be referred to an “initiating dose.” In some aspects, the initiating dose of TM buprenorphine is a means for initiating rapid induction of buprenorphine according to the disclosed methods.

In various aspects of embodiments of the disclosure, a first dose of BUP-XR is administered in an amount ranging from about 100 mg to about 300 mg, such as about 100 mg or about 300 mg, preferably about 300 mg, at least 1 hour and no more than 24 hours after administration of TM buprenorphine hydrochloride. The day on which the first dose of BUP-XR may be referred to as “day 1.”

In various aspects of embodiments of the disclosure, a second dose of BUP-XR in an amount ranging from about 100 mg to about 300 mg, such as about 100 mg or about 300 mg, preferably about 300 mg, is administered about one month, for example about 28 to 31 days, after injection of the first dose of BUP-XR.

In various aspects of embodiments of the disclosure, one or more monthly doses of BUP-XR is administered in an amount ranging from about 100 mg to about 300 mg, such as about 100 mg or about 300 mg, starting with a third dose of BUP-XR that is administered about one month, for example about 28 to 31 days, after the second dose of BUP-XR.

In various aspects of embodiments of the disclosure, BUP-XR can be administered by injection. In various embodiments, BUP-XR comprises buprenorphine free base or a pharmaceutically acceptable salt thereof. In some embodiments, BUP-XR is preferably administered by subcutaneous injection of buprenorphine free base.

For example, in some embodiments, the disclosure relates to methods of treating OUD in a human in need thereof, the methods comprising:
a) administering a composition comprising about 4 mg of TM BUP (e.g. SUBOXONE® or any other commercially available equivalent product containing buprenorphine) to the human on day 1, and
b) administering about 300 mg of BUP-XR (e.g. SUBLOCADE® or any other commercially available equivalent monthly product such as BRIXADI®) by subcutaneous injection at least about 1 hour later.

In other embodiments, the disclosure provides methods of treating OUD in a human in need thereof, the methods comprising:
a) administering a composition comprising about 4 mg of TM BUP to the human on day 1,
b) administering about 300 mg of BUP-XR by subcutaneous injection 1 at least about 1 hour later,
c) administering a second dose of about 300 mg of BUP-XR to said human by subcutaneous injection 2 as early as about day 8 or up to about one month after injection 1, and
d) administering from about 100 mg to about 300 mg of BUP-XR monthly to the human, e.g. about 100 mg or about 300 mg, by subcutaneous injection 3 starting about one about month from administration of injection 2 and continuing approximately monthly as long as needed.

In various embodiments, the RI methods described herein provide at least one of the following results:
(i) completion of steps (a) through (b) has a retention rate superior to SI at an endpoint of injection 2;
(ii) completion of steps (a) though (b) does not increase POW relative to SI;
(iii) for patients experiencing POW:
   (iii)(a) retention at injection 1 is higher after completion of step (a) with RI than for SI;
   (iii)(b) retention at injection 2 is higher after completion of steps (a) through (b) than for SI; and/or
   (iii)(c) retention at injection 3 is higher after completion of steps (a) through (c) than for SI;
(iv) the Clinical Opiate Withdrawal Score (“COWS”) from completion of steps (a) through (b) during RI is about the same as or lower than for SI;
(v) treatment-emergent adverse events (“TEAEs”) up to the second injection in steps (a) through (b) are comparable to the TEAEs up to the second injection for SI; and/or
(vi) treatment-emergent adverse events (TEAEs) up to the third injection in steps (a) through (c) are comparable to the TEAEs up to the third injection for SI.

Thus, the disclosure provides RI dosing regimens, as well as treatment methods that reduce the risk of treatment failure compared to current dosing regimens and treatment methods using buprenorphine.

The present disclosure may be understood more readily by reference to the following detailed description and examples. Unless defined otherwise, all technical and scientific terms used herein have the meaning commonly understood by one of ordinary skill in the relevant art.

### BRIEF DESCRIPTION OF THE DRAWINGS

FIGS. 1A-1B are mean (±SD) buprenorphine plasma concentration-time profiles in 300 mg/100 mg (1A) and 300 mg/300 mg (1B) dosing regimens. Both dosing regimens start with 2 monthly injections of 300 mg followed by 4 monthly injections of either 100 mg (1A) or 300 mg (1B).

FIG. 2 shows a study design for comparing the efficacy, safety, and tolerability of 100 mg and 300 mg maintenance doses of BUP-XR in treatment-seeking participants with moderate to severe OUD and high-risk opioid use. This study included an open-label sub-study comparing rapid induction vs. standard induction onto BUP-XR.

FIG. 3 shows COWS scores on induction day of the sub-study (interim data analysis).

FIG. 4 is a graph showing time to treatment discontinuation in the sub-study (final data analysis).

FIG. 5 shows COWS scores on induction day of the sub-study (final data analysis).

### DESCRIPTION

The disclosure relates to methods of treating OUD in a human patient by rapid buprenorphine induction with administration of TM buprenorphine followed by administration of a first dose of BUP-XR, followed, in some embodiments, by a second dose of BUP-XR, and optionally followed by one or more monthly maintenance doses of BUP-XR. The methods may improve treatment retention, including in individuals experiencing POW. For example, treatment retention may be improved with RI methods by at least about 10%, such as at least about 12%, or at least about 14%, for example about 10-15%, compared to SI.

The disclosed methods achieve and maintain target therapeutic plasma buprenorphine concentrations faster compared to methods employing SoC or SI induction dosing regimens, improving induction and retention success rates in BUP-XR treatment. As discussed above, BUP induction is the first step in BUP treatment. The disclosed methods achieve target therapeutic buprenorphine plasma concentrations approximately twice or even three times as quickly compared to SI. In addition, target therapeutic buprenorphine plasma concentrations may be reached at least five times as quickly, such as at least six times, at least seven times, or at least eight times as quickly using RI compared to current SoC buprenorphine induction methods. Maintained target buprenorphine concentrations are typically achieved with the second dose of BUP-XR, which is administered, for example from 8-21, 8-20, 8-19, 8-18, 8-17, 8-16, 8-15, 8-14, 8-13, 8-12, 8-11, 8-10, or 8-9 days after administration of the first initiating dose of TM BUP, in the methods described herein. In contrast, in the described SI methods of buprenorphine induction, the second dose of BUP-XR is not administered earlier than 15 days after administration of the first initiating dose of TM BUP, and in current SoC methods, the second dose of BUP-XR is administered anywhere from 38-45 after administration of the first initiating dose of TM BUP.

The dosing regimens and treatment methods disclosed achieve this goal by administering an initiating dose of TM BUP and, within 24 hours, administering a first dose of BUP-XR, followed by a second dose of BUP-XR within about 2 to about 31 days, preferably about one week, after the first dose of BUP-XR. It was found that this dosing regimen was safe and resulted in patients having a reduced time below target plasma buprenorphine levels, thus helping to control symptoms of craving and withdrawal. The disclosed dosing regimens can also include one or more additional doses of BUP-XR starting with a third dose about a month after the second dose, and continue monthly thereafter as needed. As discussed in more detail below, the disclosed RI dosing regimens and treatment methods surprisingly resulted in a greater number of patients completing induction and receiving the second and third doses of BUP-XR compared to SI.

In various aspects, the patient is one who uses opioids via an injection route. In various aspects, the patient is from high-risk populations. In various aspects, the patient is one who uses intravenous drugs, very high doses of opioids, or highly potent synthetic opioids. Those using very high doses or highly potent synthetic opioids can have an increased risk of treatment failure and opioid overdose. In aspects, the patient is black or African American who self-identifies their race as black or African American. In aspects, the patient is white who self-identifies their race as white. In aspects, the patient is Hispanic who self-identifies their race as Hispanic. In aspects, the patient is Asian who self-identifies their race as Asian. In aspects, the patient is a combination of 2 or more races who self-identify their race as the combination of 2 or more races.

In various aspects, the methods comprise administering about 2 mg to about 4 mg of TM buprenorphine on day 1 of treatment, followed by subcutaneous administration of about 100 mg to about 300 mg of BUP-XR (injection 1) within no more than about 24 hours, for example within about 1 hour to about 12 hours, within about 1 hour to about 6 hours, or within about 1 hour to about 3 hours. The methods may further comprise administration of a second subcutaneous injection of about 100 mg to about 300 mg of BUP-XR (injection 2) approximately one week later, e.g. on one of days 8-12, such as day 8. In various aspects, the methods further comprise monthly subcutaneous administration of about 100 mg to about 300 mg of BUP-XR (injections 3+) for a prescribed time period.

For example, the methods may comprise administering about 2 mg to about 4 mg of TM buprenorphine on day 1 of treatment, followed by subcutaneous administration of about 300 mg of BUP-XR (injection 1) within no more than about 24 hours, for example within about 1 hour to about 12 hours, within about 1 hour to about 6 hours, or within about 1 hour to about 3 hours. The methods may optionally further comprise administration of a second subcutaneous injection of about 100 mg to about 300 mg of BUP-XR (injection 2) approximately one week later, e.g. on one of days 8-12, such as day 8.

As a further example, the methods may comprise administering about 2 mg to about 4 mg of TM buprenorphine on day 1 of treatment, followed by subcutaneous administration of about 100 mg to about 300 mg of BUP-XR (injection 1) within no more than about 24 hours, no more than about 12 hours, no more than about 8 hours, no more than about 6 hours, or no more than about 3 hours, for example within about 1 hour to about 12 hours, within about 1 hour to about 6 hours, or within about 1 hour to about 3 hours, such as within about 1 hour; administering a second subcutaneous injection of about 100 mg to about 300 mg of BUP-XR (injection 2) approximately one week later, e.g. on one of days 8-12, such as day 8; and administering one or more monthly subcutaneous injections of about 100 mg to about 300 mg of BUP-XR (injections 3+) for a prescribed time period.

As yet a further example, the methods may comprise administering about 2 mg to about 4 mg of TM BUP, e.g., 4 mg, on day 1 of treatment, followed by administration of a first dose of BUP-XR, e.g., subcutaneous injection of about 300 mg of BUP-XR (injection 1), within no more than about 24 hours, for example within about 1 hour to about 12 hours, within about 1 hour to about 6 hours, or within about 1 hour to about 3 hours; administering a second dose of BUP-XR, e.g., subcutaneous injection of about 100 mg to about 300 mg of BUP-XR (injection 2) approximately one week later, e.g. on one of days 8-12, such as day 8; and administering one or more monthly doses of BUP-XR, e.g., subcutaneous injections of about 100 mg or about 300 mg of BUP-XR (injections 3+) for a prescribed time period.

In various embodiments, the total number of doses of BUP-XR administered to the patient in the methods according to the disclosure may be 1, or may be more than 1. By way of example only, the total number of doses of BUP-XR in the methods according to the disclosure may range from 1 to 10, from 1 to 9, from 1 to 8, from 1 to 7, from 1 to 6, from 1 to 5, from 1 to 4, from 1 to 3, from 1 to 2, from 2 to 10, from 2 to 9, from 2 to 8, from 2 to 7, from 2 to 6, from 2 to 5, from 2 to 4, from 2 to 3, from 3 to 10, from 3 to 9, from 3 to 8, from 3 to 7, from 3 to 6, from 3 to 5, or from 3 to 4.

As used herein, the term “BUP” refers to buprenorphine.

The terms “extended-release buprenorphine” and “BUP-XR” refer to a buprenorphine formulation that is formulated to last longer in the body than immediate-release buprenorphine, so that the intended benefit lasts over a period of time of about one month. Thus, BUP-XR is understood as being formulated for monthly administration. As non-limiting examples, SUBLOCADE® and RBP-6000, in which the active ingredient is buprenorphine free base, may be used. In some aspects, BUP-XR contains about 300 mg buprenorphine free base. In other aspects, BUP-XR contains about 100 mg buprenorphine free base. In some aspects, a composition comprising about 100 mg BUP-XR and/or a composition comprising about 300 mg BUP-XR comprises (i) about 18 wt % of buprenorphine free base; (ii) about 32 wt % of a poly(DL-lactide-co-glycolide) copolymer; and (iii) about 50 wt % of N-methyl-2-pyrrolidone. In other aspects, BUP-XR can be any commercially available extended-release product having buprenorphine free base or a salt thereof as the active ingredient.

In various aspects, the BUP-XR formulations comprise about 300 mg of buprenorphine, which is defined as 295 mg to 305 mg.

In various aspects, BUP-XR formulations comprise about 100 mg of buprenorphine, defined as 95 mg to 105 mg.

In various aspects, about 18 wt % of buprenorphine free base is defined as from 17 wt % to 19 wt %; about 32 wt % of a poly(DL-lactide-co-glycolide) copolymer is defined as from 31 wt % to 33 wt %; and about 50 wt % of N-methyl-2-pyrrolidone is defined as from 48 wt % to 52 wt %.

As used herein, “day 1” refers to the day on which dose 1 of BUP-XR is administered. The second dose of BUP-XR is intended to be administered approximately one week later, e.g., on day 8. However, it should be understood that if the second dose is not given on day 8, rapid induction can still be achieved if the second dose is given from 2 days to less than one month after the first dose, for example from days 4-21 such as from days 9-12.

A dosing of BUP-XR that is “monthly” means that the BUP-XR is administered one time in one month, and at least a second time one month later. “One month” is defined as about 28 days to about 31 days. For example, the BUP-XR can be administered the first time on January 1, the second time on February 1, the third time on March 1, etc. The terms “monthly” and “4 weeks” may be used interchangeably herein.

As used herein, “TM BUP” and “TM buprenorphine” refer to transmucosal buprenorphine formulations, such as Indivior's product SUBOXONE® that contains buprenorphine hydrochloride and naloxone hydrochloride.

It is understood that amounts of buprenorphine and naloxone herein are given with reference to the amount of free base. For example, reference to “a 4 mg dose of TM buprenorphine hydrochloride” is understood as an amount of buprenorphine hydrochloride equivalent to 4 mg of buprenorphine free base (e.g., about 4.31-4.32 mg buprenorphine hydrochloride).

With reference to the Diagnostic and Statistical Manual for Mental Disorders, 5th Edition, American Psychiatric Association, 2013 (also referred to herein as DSM5), “opioid use disorder” is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. In aspects, the opioid use disorder is moderate opioid use disorder. “Moderate opioid use disorder” is defined by reference to the DSM5 Opioid Use Disorder Checklist (ICD-9-CM code 304.00 or ICD-10-CM code F11.20) as having the presence of 4 or 5 symptoms indicated in the DSM5 Opioid Use Disorder Checklist. In aspects, the opioid use disorder is severe opioid use disorder. “Severe opioid use disorder” is defined by reference to the DSM5 Opioid Use Disorder Checklist (ICD-9-CM code 304.00 or ICD-10-CM code F11.20) as having the presence of 6 or more symptoms indicated in the DSM5 Opioid Use Disorder Checklist. In aspects, the opioid use disorder is moderate-to-severe opioid use disorder. Moderate-to-severe opioid use disorder refers to the presence of 4 or more symptoms indicated in the DSM5 Opioid Use Disorder Checklist. In aspects, the opioid use disorder is mild opioid use disorder. “Mild opioid use disorder” is defined by reference to the DSM5 Opioid Use Disorder Checklist (ICD-9-CM code 305.50 or ICD-10-CM code F11.10) as having the presence of 2 or 3 symptoms indicated in the DSM5 Opioid Use Disorder Checklist. In aspects, the opioid use disorder is mild-to-moderate opioid use disorder. Mild-to-moderate opioid use disorder refers to the presence of 2 to 5 symptoms indicated in the DSM5 Opioid Use Disorder Checklist (www.asam.org/docs/default-source/education-docs/dsm-5-dx-oud-8-28-2017.pdf). In aspects, “treating opioid use disorder” encompasses one or more of (i) reducing opioid withdrawal symptoms, (ii) eliminating opioid withdrawal symptoms, (iii) reducing opioid craving, (iv) eliminating opioid craving, (v) reducing illicit opioid use, (vi) eliminating illicit opioid use, and (vii) inducing opioid abstinence. For example, a reduction in the number of times a patient uses opioids (e.g. as calculated on a daily or weekly basis) would be considered effective treatment of OUD, even if total abstinence is not achieved.

The term “opioid withdrawal signs and symptoms” includes one or more signs and symptoms associated with withdrawal from opioids. Such signs and symptoms can include one or more of the following: agitation, anxiety, muscle aches, increased tearing, insomnia, runny nose, sweating, yawning, abdominal cramping, diarrhea, dilated pupils, goose bumps, nausea, and vomiting. Opioid withdrawal symptoms can begin to occur from a few hours to a few days after the last intake of an opioid, with the time being dependent on the opioid, the person's metabolism, and other factors.

As used herein, “improving treatment retention” and variations thereof means that an individual's likelihood of remaining in buprenorphine treatment through administration of the second dose and/or the third dose of BUP-XR is increased compared to SI methods of buprenorphine induction.

The term “administering” includes administration of the formulations described herein to the patient, and can include implantation of a slow-release device in the patient. In aspects, “administering” a dose of BUP-XR refers to parenteral administration, for example subcutaneously injecting the formulations. Parenteral administration includes, for example, intravenous, intramuscular, intra-arterial, intradermal, intrathecal, subcutaneous, intraperitoneal, intraventricular, or intracranial. In aspects, “administering” TM BUP refers to buccal or sublingual administration. However, it is understood that the term “administering” (and variants thereof) includes but is not intended to be limited to physically providing or administering a formulation to a patient. For example, “administering TM buprenorphine” can include prescribing TM buprenorphine to a patient wherein the patient obtains the product from a pharmacy and places the film under their tongue.

As used herein, the terms “patient” and “subject” refer to a human.

A “high-risk” patient includes those that use intravenous drugs, very high doses of opioids, or highly potent synthetic opioids such as fentanyl. A high-risk patient may also have a long history of drug-use, for example at least about 5, at least about 6, at least about 7, at least about 8, at least about 9, at least about 10, at least about 11, at least about 12, at least about 13, at least about 14, or at least about 15 years of drug use, may have had one or more previous overdoses that required medical assistance such as an emergency department visit, and/or may have previously had unsuccessful OUD treatment, for example with buprenorphine, methadone, or naltrexone.

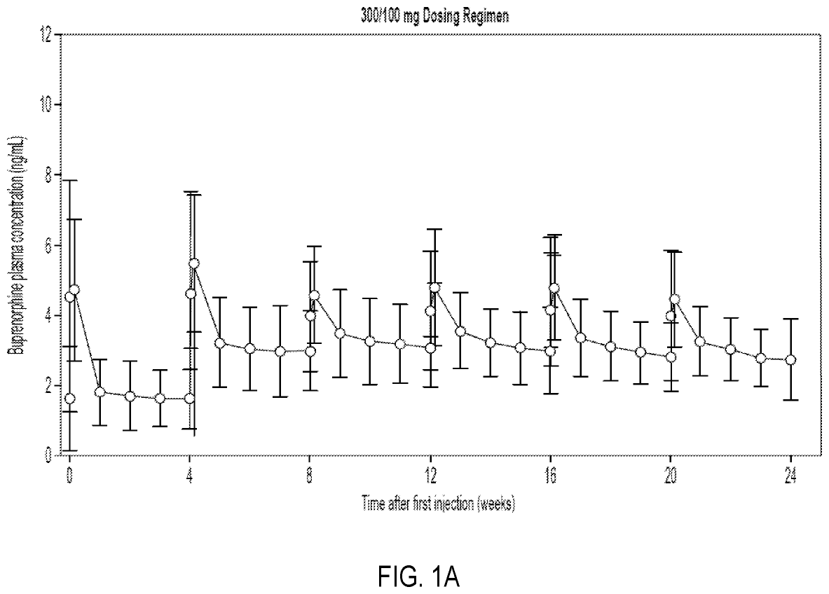

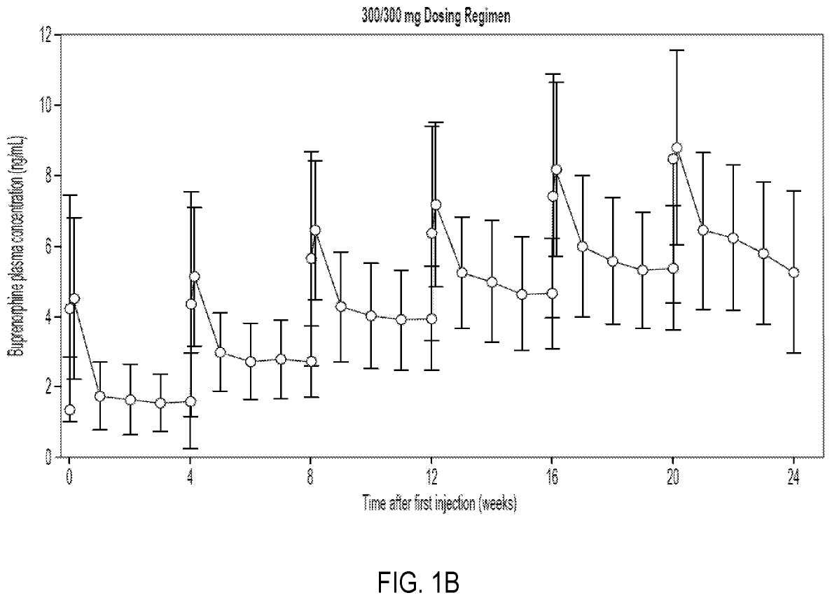

“Therapeutic levels” of buprenorphine refers to buprenorphine plasma concentrations that can be effective to achieve at least one of the following results: (a) treatment of OUD; (b) suppressing opioid withdrawal symptoms; (c) eliminating opioid withdrawal symptoms; (d) reducing opioid craving; (e) eliminating opioid craving; (f) reducing illicit opioid use; (g) preventing illicit opioid use; (h) inducing opioid abstinence; or (i) a combination of two or more of the foregoing. “Therapeutic levels” of buprenorphine are generally greater than 1.5 ng/mL, for example at least about 1.75 ng/mL or greater, or at least about 2 ng/mL or greater (see FIGS. 1A-1B). “Therapeutic levels” can also be described in terms of steady-state minimum buprenorphine plasma concentration levels, steady-state average buprenorphine plasma concentration levels, and steady-state maximum buprenorphine plasma concentration levels.

As used herein, “maintaining target buprenorphine plasma concentration levels” and variations thereof means that the lowest concentration before the next monthly dose does not fall below the therapeutic threshold level.

Exemplary embodiment 1: A method of improving patient retention in OUD treatment with BUP-XR, the method comprising (a) on day 1, administering to said patient an initiating dose of TM BUP and, within less than about 12 hours, such as less than about 6 hours, for example about 1-3 hours, subsequently administering to said patient a first extended release composition comprising about 300 mg of buprenorphine, and (b) on one of days 8-21, administering to said patient a second extended release composition comprising about 300 mg of buprenorphine, wherein patient retention is improved compared to standard induction of buprenorphine induction.

Exemplary embodiment 2: The method according to exemplary embodiment 1, wherein the second composition is administered to the patient on one of days 8-12, such as one of days 8-10.

Exemplary embodiment 3: The method according to either of exemplary embodiments 1 or 2, wherein the initiating dose comprises about 2 mg or about 4 mg of TM BUP.

Exemplary embodiment 4: The method according to any of exemplary embodiments 1-3, wherein the initiating dose comprises about 4 mg of TM BUP and about 1 mg of naloxone.

Exemplary embodiment 5: The method according to any of exemplary embodiments 1-4, wherein the first extended release composition and/or the second extended release composition further comprises about 32 wt % of a poly(DL-lactide-co-glycolide) copolymer and/or about 50 wt % of N-methyl-2-pyrrolidone.

Exemplary embodiment 6: The method according to any of exemplary embodiments 1-5, further comprising (c) administering a third extended release composition comprising about 100 mg or about 300 mg of buprenorphine to the patient about one month after administration of the second extended release composition, and optionally (d) administering one or more monthly extended release composition comprising about 100 mg or about 300 mg of buprenorphine to the patient starting about one month after administration of the third extended release composition.

Exemplary embodiment 7: The method according to any of exemplary embodiments 1-6, wherein the total number of doses of extended release compositions administered to the patient is at least 2, such as at least 3 or at least 4, for example ranges from 2 to 10, from 3 to 10, or from 4 to 10.

Exemplary embodiment 8: The method according to any of exemplary embodiments 1-7, wherein on day 1, at least one additional dose of TM BUP is administered to the patient before and/or after administration of the first extended release composition.

Exemplary embodiment 9: The method according to any of exemplary embodiments 1-8, wherein said improvement is statistically significant.

Exemplary embodiment 10: The method according to any of exemplary embodiments 1-9, wherein the improvement is greater than 10% compared to SI.

Exemplary embodiment 11: The method according to any of exemplary embodiments 1-10, wherein the patient is fentanyl-positive by self-reporting or drug screening on day 1.

Exemplary embodiment 12: The method according to any of exemplary embodiments 1-11, wherein the patient has had one or more previous overdoses requiring medical treatment.

Exemplary embodiment 13: The method according to any of exemplary embodiments 1-12, wherein the patient has used drugs for at least about 10 years and/or has previously received medical treatment for OUD.

Exemplary embodiment 14: The method according to any of exemplary embodiments 1-13, wherein the patient experiences precipitated opioid withdrawal during said treatment with BUP-XR.

Exemplary embodiment 15: A method of achieving and maintaining a target therapeutic buprenorphine plasma concentration in OUD treatment with BUP-XR at least twice as quickly as with SI, the method comprising (a) on day 1, administering to said patient an initiating dose of TM BUP and, within less than about 12 hours, such as less than about 6 hours, for example about 1-3 hours, subsequently administering to said patient a first extended release composition comprising about 300 mg of buprenorphine, and (b) on one of days 8-21, administering to said patient a second extended release composition comprising about 300 mg of buprenorphine, wherein said target therapeutic buprenorphine plasma concentration is greater than about 1.5 ng/mL, such as greater than about 1.75 ng/mL, or greater than about 2 ng/mL.

Exemplary embodiment 16: The method according to exemplary embodiment 15, wherein the second composition is administered to the patient on one of days 8-14.

Exemplary embodiment 17: The method according to either of exemplary embodiments 15 or 16, wherein the second composition is administered to the patient on one of days 8-12, such as day 8.

Exemplary embodiment 18: The method according to any of exemplary embodiments 15-17, wherein the initiating dose comprises about 2 mg or about 4 mg of TM BUP.

Exemplary embodiment 19: The method according to any of exemplary embodiments 15-18, wherein the initiating dose comprises about 4 mg of TM BUP and about 1 mg of naloxone.

Exemplary embodiment 20: The method according to any of exemplary embodiments 15-19, wherein the first extended release composition and/or the second extended release composition further comprises about 32 wt % of a poly(DL-lactide-co-glycolide) copolymer and/or about 50 wt % of N-methyl-2-pyrrolidone wherein the first extended release composition and/or the second extended release composition further comprises about 32 wt % of a poly(DL-lactide-co-glycolide) copolymer and/or about 50 wt % of N-methyl-2-pyrrolidone.

Exemplary embodiment 21: The method according to any of exemplary embodiments 15-20, wherein on day 1, at least one additional dose of TM BUP is administered to the patient before and/or after administration of the first extended release composition.

Exemplary embodiment 22: The method according to any of exemplary embodiments 15-21, wherein said target therapeutic buprenorphine plasma concentration is achieved at least three times as quickly as with SI.

Exemplary embodiment 23: The method according to any of exemplary embodiments 15-22, wherein the patient is fentanyl-positive by self-reporting or drug screening on day 1.

Exemplary embodiment 24: The method according to any of exemplary embodiments 15-23, wherein the patient has had one or more previous overdoses requiring medical treatment.

Exemplary embodiment 25: The method according to any of exemplary embodiments 15-24, wherein the patient has used drugs for at least about 10 years and/or has previously received medical treatment for OUD.

Exemplary embodiment 26: A method of achieving and maintaining a target therapeutic buprenorphine plasma concentration in OUD treatment with BUP-XR at least five times as quickly as with current SoC methods, the method comprising (a) on day 1, administering to said patient an initiating dose of TM BUP and, within less than about 12 hours, such as less than about 6 hours, for example about 1-3 hours, subsequently administering to said patient a first extended release composition comprising about 300 mg of buprenorphine, and (b) on one of days 8-21, administering to said patient a second extended release composition comprising about 300 mg of buprenorphine, wherein said target therapeutic buprenorphine plasma concentration is greater than about 1.5 ng/mL, such as greater than about 1.75 ng/mL, or greater than about 2 ng/mL.

Exemplary embodiment 27: The method according to exemplary embodiment 26, wherein the second composition is administered to the patient on one of days 8-14.

Exemplary embodiment 28: The method according to either of exemplary embodiments 26 or 27, wherein the second composition is administered to the patient on one of days 8-12, such as day 8.

Exemplary embodiment 29: The method according to any of exemplary embodiments 26-28, wherein the initiating dose comprises about 2 mg or about 4 mg of TM BUP.

Exemplary embodiment 30: The method according to any of exemplary embodiments 26-29, wherein the initiating dose comprises about 4 mg of TM BUP and about 1 mg of naloxone.

Exemplary embodiment 31: The method according to any of exemplary embodiments 26-30, wherein the first extended release composition and/or the second extended release composition further comprises about 32 wt % of a poly(DL-lactide-co-glycolide) copolymer and/or about 50 wt % of N-methyl-2-pyrrolidone wherein the first extended release composition and/or the second extended release composition further comprises about 32 wt % of a poly(DL-lactide-co-glycolide) copolymer and/or about 50 wt % of N-methyl-2-pyrrolidone.

Exemplary embodiment 32: The method according to any of exemplary embodiments 26-31, wherein on day 1, at least one additional dose of TM BUP is administered to the patient before and/or after administration of the first extended release composition.

Exemplary embodiment 33: The method according to any of exemplary embodiments 26-32, wherein said target therapeutic buprenorphine plasma concentration is achieved at least six times as quickly as with current SoC methods.

Exemplary embodiment 34: The method according to any of exemplary embodiments 26-33, wherein the patient is fentanyl-positive by self-reporting or drug screening on day 1.

Exemplary embodiment 35: The method according to any of exemplary embodiments 26-34, wherein the patient has had one or more previous overdoses requiring medical treatment.

Exemplary embodiment 36: The method according to any of exemplary embodiments 26-35, wherein the patient has used drugs for at least about 10 years and/or has previously received medical treatment for OUD.

Exemplary embodiment 37: Rapid induction of buprenorphine treatment comprising (a) on day 1, administering to said patient an initiating dose of TM BUP and, within less than about 12 hours, such as less than about 6 hours, for example about 1-3 hours, subsequently administering to said patient a first extended release composition comprising about 300 mg of buprenorphine, and (b) on one of days 8-21, administering to said patient a second extended release composition comprising about 300 mg of buprenorphine, wherein patient retention is improved compared to standard induction of buprenorphine induction.

Exemplary embodiment 38: Rapid induction according to exemplary embodiment 37, wherein the second composition is administered to the patient on one of days 8-14, such as days 8-12 or days 8-10.

Exemplary embodiment 39: Rapid induction according to either of exemplary embodiments 37 or 38, wherein the initiating dose comprises about 2 mg or about 4 mg of TM BUP.

Exemplary embodiment 40: Rapid induction according to any of exemplary embodiments 37-39, wherein the initiating dose comprises about 4 mg of TM BUP and about 1 mg of naloxone.

Exemplary embodiment 41: Rapid induction according to any of exemplary embodiments 37-40, wherein the first extended release composition and/or the second extended release composition further comprises about 32 wt % of a poly(DL-lactide-co-glycolide) copolymer and/or about 50 wt % of N-methyl-2-pyrrolidone.

Exemplary embodiment 42: Rapid induction according to any of exemplary embodiments 37-41, wherein on day 1, at least one additional dose of TM BUP is administered to the patient before and/or after administration of the first extended release composition.

Exemplary embodiment 43: Rapid induction according to any of exemplary embodiments 37-42, wherein the patient is fentanyl-positive by self-reporting or drug screening on day 1.

Exemplary embodiment 44: Rapid induction according to any of exemplary embodiments 37-43, wherein the patient has had one or more previous overdoses requiring medical treatment.

Exemplary embodiment 45: Rapid induction according to any of exemplary embodiments 37-44, wherein the patient has used drugs for at least about 10 years and/or has previously received medical treatment for OUD.

Exemplary embodiment 46: A method of reducing COWS during rapid induction of buprenorphine treatment comprising administering an initiating dose of TM BUP to a patient and, within less than about 12 hours, such as less than about 6 hours, for example about 1-3 hours, subsequently administering to said patient an extended release composition comprising about 300 mg of buprenorphine.

Exemplary embodiment 47: The method according to exemplary embodiment 46, wherein the patient is fentanyl-positive by self-reporting or drug screening.

### Example

The following non-limiting Example describes a study comparing RI and SI for induction of monthly injectable BUP-XR in treatment-seeking adult participants with OUD and high-risk opioid use (NCT04995029). The study demonstrates the surprising increased success in buprenorphine treatment of OUD achieved with RI by comparing the efficacy, safety, and tolerability of two maintenance dose levels of BUP-XR, 300 mg and 100 mg, administered every 4 weeks, in treatment-seeking participants with moderate to severe OUD and high-risk opioid use (those who use opioids via an injection route and/or use high doses of opioids and/or use highly potent synthetic opioids) that may benefit from the higher 300 mg maintenance dose.

In the study, buprenorphine plasma concentrations for two dosing regimens of BUP-XR were evaluated. The first regimen shown in FIG. 1A (n=203) consisted of two monthly injections of 300 mg BUP-XR followed by four monthly injections of 100 mg BUP-XR. The second regimen shown in FIG. 1B (n=201) involved six monthly injections of 300 mg BUP-XR. The data in FIGS. 1A-1B were analyzed and presented with standard deviation (SD) to account for variability in the results.

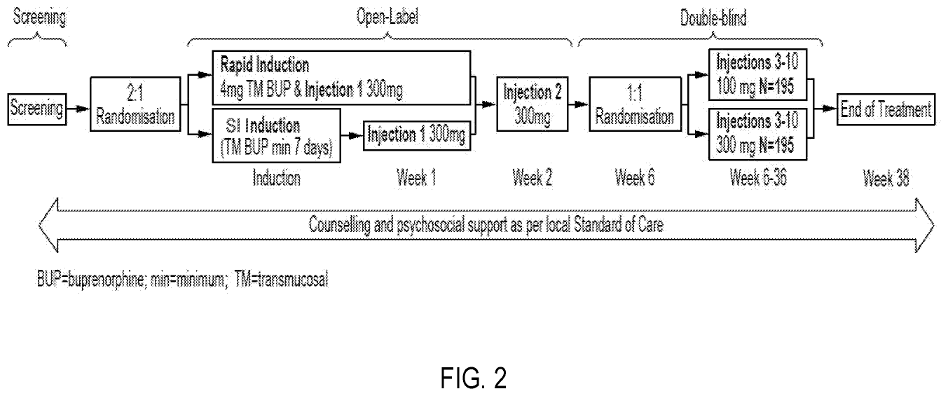

The study design is shown in FIG. 2. In FIG. 2, week 1 is time zero (i.e. there is no week 0), so week 6 is 5 weeks after day 1. Participants were randomized at a 2:1 ratio to the RI or SI arms. Either SUBOXONE® (4 mg buprenorphine hydrochloride/1 mg naloxone hydrochloride) or another transmucosal product was used for the TM BUP initiating dose in the study, and was administered either under the tongue (sublingual) or between the gum and cheek (buccal). SUBLOCADE® was used as BUP-XR for the study.

Rapid Induction: The initial subcutaneous injection of BUP-XR occurred on the same day as administration of TM BUP for the RI arm. First, 4 mg of TM BUP was administered to the participants on day 1, with an additional one or two doses of TM BUP administered if needed. After monitoring the subject for approximately one hour, 300 mg of BUP-XR by subcutaneous injection (“injection 1” or “first dose”) was administered. A subsequent dose of 300 mg of BUP-XR was by subcutaneous (“injection 2” or “second dose”) one week after injection 1.

Standard Induction: In the SI arm, TM BUP was administered for a minimum of 7 days as per applicable product labelling information. In some cases, TM BUP was administered for 7-14 days. Subsequently, 300 mg of BUP-XR was administered to the SI participants meeting eligibility requirements by subcutaneous injection (injection 1). In SI, injection 1 occurred after at least 7 days of induction and dose stabilization with TM BUP. The second dose of 300 mg of BUP-XR was by subcutaneous injection 2 one week after injection 1.

As shown in FIGS. 1A, 1B, and 2, subcutaneous injections 3-10 of 100 mg or 300 mg of BUP-XR were then administered on an approximately monthly or 4-week basis for both the RI and SI arms.

In the study, day 1 is the time of injection 1 for both groups (RI and SI); day 8 is the time of injection 2 (8=1+7); and day 36 is the time of injection 3 (36=1+7+28). Because injection 1 in the RI group was administered on the same day as TM BUP, subjects in the RI group received injection 1 anywhere from 7-14 days earlier than subjects in the SI group.

The study also included an open-label induction sub-study (“the sub-study”), the primary objective of which was to compare treatment retention of participants initiated onto BUP-XR using RI compared to SI.

### Eligibility

Eligible participants were selected based on the inclusion criteria described hereafter during the screening. All eligible participants signed the informed consent form (ICF) and were able and willing to comply with the requirements and restrictions described hereafter. Proper counselling and psychosocial support as per the local standards were provided to the patients. Eligible participants were selected based on the following inclusion criteria during screening: (1) had signed the ICF and was willing and able to comply with the requirements and restrictions therein; (2) was 18 years of age or older at the time of signing the ICF; (3) currently met DSM-5 criteria for moderate or severe OUD; (4) had a history of OUD as defined by documented medical history of OUD for at least 90 days immediately prior to providing informed consent; (5) met at least one of the following criteria for high-risk opioid use at the screening visit: (a) using opioids via the injection route for an average of 5 or more days per week in the last 4 weeks; or (b) using at least 500 mg IV heroin equivalent (e.g., 1250 mg intravenous (IV) morphine) or self-reported use of any dose of highly potent synthetic opioids (fentanyl and analogues excluding transdermal patches) for an average of 5 or more days per week in the last 4 weeks by any route; (6) was seeking medication for the treatment of OUD; (7) was an appropriate candidate for opioid partial-agonist medications for OUD in the opinion of the Investigator or medically qualified sub-Investigator; (8) agreed not to use buprenorphine-containing products other than those administered as part of study treatment for the duration of the treatment period; and (9) if female, was not pregnant, not lactating and, if of childbearing potential, agreed not to become pregnant while in the study and to use medically acceptable means of contraception while in the study.

Participants were not eligible for inclusion in the study if any of the following exclusion criteria applied: (1) had a current diagnosis, other than OUD, requiring chronic opioid treatment; (2) has a concurrent primary substance use disorder, as defined by DSM-5 criteria, other than opioid, tobacco, cannabis, or alcohol use disorders; (3) met DSM-5 criteria for severe alcohol use disorder; (4) had significant traumatic injury or major surgical procedure (as defined by the Investigator) within 30 days prior to the first dose of BUP-XR or still recovering from prior injury or surgery; (5) had congenital long QT syndrome, history of prolonged QT in the 3 months prior to screening, or history of medications or other factors that are at risk for Torsades de Pointes; (6) had abdominal area unsuitable for sub-cutaneous injections (e.g., nodules, scarring, lesions, excessive pigment, etc.); (7) had a history of suicidal ideation within 30 days prior to informed consentor history of a suicide attempt in the 6 months prior to informed consent; (8) had uncontrolled intercurrent illness including, but not limited to, a medical or psychiatric illness/social situation that would limit compliance with study requirements or compromise the ability of the participant to provide written informed consent; (9) had any other active medical condition, organ disease, or concurrent medication or treatment that may either compromise participant safety or interfere with study endpoints; (10) had total bilirubin of ≥1.5× upper limit of normal (“ULN”) (with direct bilirubin >1.3 mg/dL), ALT≥3×ULN, AST≥3×ULN, serum creatinine>2×ULN, or international normalized ratio (“INR”)>1.5×ULN at screening; (11) had known allergy or hypersensitivity to buprenorphine or any component of the ATRIGEL delivery system; (12) was undergoing concurrent treatment, or has had prior treatment, with any long-acting form of a buprenorphine-containing product in the past 2 years, or if >2 years had a positive urine drug screen (“UDS”) for buprenorphine at screening; treatment with oral buprenorphine, oral naltrexone, or methadone products within 14 days prior to consent; or treatment with depot naltrexone within 3 months prior to consent; (13) was undergoing concurrent treatment with another investigational agent or enrollment in another clinical study (except for an observational study) or treatment with another investigational agent within 30 days prior to screening; (14) was undergoing concurrent treatment with medications contraindicated for use with buprenorphine as per local prescribing information; (15) was under court order requiring treatment for OUD; (16) was a member of site staff and/or has a financial interest in the sponsor, or is an immediate family member of either the site staff and/or employee of the sponsor, directly involved in the study; or (17) had any pending legal status or pending legal action that could prohibit full participation in or compliance with study procedures.

A total of 1434 participants were screened and 785 were randomized. The top reasons for screen failure included 410 participants (28.6%) who were unable to comply with study requirements and did not return after screening, 49 participants (3.4%) with liver function test issues, and 39 participants (2.7%) who did not meet the criteria for high-risk opioid users. Table 1 shows the disposition of all participants screened.

TABLE 1
Disposition of Participants

Overall

| | |
|---|---|
| SI | RI |

| | |
|---|---|
| Screened | 1434 |
| Randomized | 785 (54.7%) |

| | |
|---|---|
| 267 | 518 |

Of the 785 randomized participants, 729 (92.9%) were included in the Safety Full Analysis Set (“SFAS”). Of the 729 participants in the SFAS, 6 non-eligible participants were discontinued after randomization. Therefore, 723 participants were evaluable for treatment retention, which formed the basis for the primary efficacy analysis. Table 2 shows the demographic and baseline characteristics of SFAS participants.

TABLE 2
Demographics and Baseline Characteristics

| Fentanyl positive | | Fentanyl negative | | Total | | |
|---|---|---|---|---|---|---|
| (n = 563) | | (n = 166) | | (N = 729)^{a} | | |
| RI | SI | RI | SI | RI | SI | Total |
| (n = 367) | (n = 196) | (n = 107) | (n = 59) | (n = 474) | (n = 255) | (N = 729) |

Demographics

| | | | | | | | |
|---|---|---|---|---|---|---|---|
| Age | 40.0 | 39.5 | 44.2 | 43.3 | 40.9 | 40.4 | 40.7 |
| mean (SD), years^{b} | (10.5) | (9.3) | (10.8) | (10.8) | (10.7) | (9.8) | (10.4) |

Sex

| | | | | | | | |
|---|---|---|---|---|---|---|---|
| Male | 215 | 106 | 57 | 28 | 272 | 134 | 406 |
| | (58.6) | (54.1) | (53.3) | (47.5) | (57.4) | (52.5) | (55.7) |
| Female | 152 | 90 | 50 | 31 | 202 | 121 | 323 |
| | (41.4) | (45.9 | (46.7) | (52.5) | (42.6) | (47.5) | (44.3) |

Race

| | | | | | | | |
|---|---|---|---|---|---|---|---|
| American Indian or | 4 | 1 | 0 | 1 | 4 | 2 | 6 |
| Alaska Native | (1.1) | (0.5) | | (1.7) | (0.8) | (0.8) | (0.8) |
| Asian | 1 | 0 | 0 | 0 | 1 | 0 | 1 |
| | (0.3) | | | | (0.2) | | (0.1) |
| Black or African | 53 | 23 | 32 | 14 | 85 | 37 | 122 |
| American | (14.4) | (11.7) | (29.9) | (23.7) | (17.9) | (14.5) | (16.7) |
| Native Hawaiian or other | 4 | 1 | 0 | 0 | 4 | 1 | 5 |
| Pacific Islander | (1.1) | (0.5) | | | (0.8) | (0.4) | (0.7) |
| White | 289 | 165 | 69 | 42 | 358 | 207 | 565 |
| | (78.7) | (84.2) | (64.5) | (71.2) | (75.5) | (81.2) | (77.5) |
| Other (multiple | 11 | 5 | 6 | 2 | 17 | 7 | 24 |
| categories reported)^{c} | (3.0) | (2.6) | (5.6) | (3.4) | (3.6) | (2.7) | (3.3) |
| Not reported | 5 | 1 | 0 | 0 | 5 | 1 | 6 |
| | (1.4) | (0.5) | | | (1.1) | (0.4) | (0.8) |

Ethnicity

| | | | | | | | |
|---|---|---|---|---|---|---|---|
| Hispanic or Latino | 66 | 26 | 10 | 6 | 76 | 32 | 108 |
| | (18.0) | (13.3) | (9.3) | (10.2) | (16.0) | (12.5) | (14.8) |
| Non-Hispanic or non- | 298 | 167 | 96 | 53 | 394 | 220 | 614 |
| Latino | (81.2) | (85.2) | (89.7) | (89.8) | (83.1) | (86.3) | (84.2) |
| Not reported | 3 | 3 | 1 | 0 | 4 | 3 | 7 |
| | (0.8) | (1.5) | (0.9) | | (0.8) | (1.2) | (1.0) |

Drug use history

| | | | | | | | |
|---|---|---|---|---|---|---|---|
| Opioid lifetime use, | 14.4 | 14.3 | 15.9 | 15.9 | 14.8 | 14.6 | 14.7 |
| mean (SD), years^{d} | (9.5) | (9.5) | (10.5) | (8.9) | (9.8) | (9.4) | (9.6) |
| Opioid use past 4 weeks, | 27.8 | 27.8 | 27.7 | 27.6 | 27.8 | 27.7 | 27.8 |
| mean (SD), days^{e} | (1.0) | (1.2) | (1.5) | (1.5) | (1.1) | (1.3) | (1.2) |

Opioids used in past 4 weeks^{f}

| | | | | | | | |
|---|---|---|---|---|---|---|---|
| Fentanyl | 329 | 175 | 44 | 24 | 373 | 199 | 572 |
| | (89.6) | (89.3) | (41.1) | (40.7) | (78.7) | (78.0) | (78.5) |
| Heroin | 172 | 100 | 92 | 51 | 264 | 151 | 415 |
| | (46.9) | (51.0) | (86.0) | (86.4) | (55.7) | (59.2) | (56.9) |
| Oxycodone | 44 | 26 | 13 | 11 | 57 | 37 | 94 |
| | (12.0) | (13.3) | (12.1) | (18.6) | (12.0) | (14.5) | (12.9) |
| Hydrocodone | 23 | 10 | 10 | 4 | 33 | 14 | 47 |
| | (6.3) | (5.1) | (9.3) | (6.8) | (7.0) | (5.5) | (6.4) |
| Methadone | 9 | 8 | 6 | 6 | 15 | 14 | 29 |
| | (2.5) | (4.1) | (5.6) | (10.2) | (3.2) | (5.5) | (4.0) |
| Morphine | 8 | 6 | 4 | 6 | 12 | 12 | 24 |
| | (2.2) | (3.1) | (3.7) | (10.2) | (2.5) | (4.7) | (3.3) |
| Other | 6 | 2 | 3 | 2 | 9 | 4 | 13 |
| | (1.6) | (1.0) | (2.8) | (3.4) | (1.9) | (1.6) | (1.8) |

Main route of opioid in past 4 weeks^{e}

| | | | | | | | |
|---|---|---|---|---|---|---|---|
| Smoking | 181 | 90 | 37 | 19 | 218 | 109 | 327 |
| | (49.3) | (45.9) | (34.6) | (32.2) | (46.0) | (42.7) | (44.9) |
| Injection | 90 | 52 | 41 | 27 | 131 | 79 | 210 |
| | (24.5) | (26.5) | (38.3) | (45.8) | (27.6) | (31.0) | (28.8) |
| Snorting | 89 | 49 | 25 | 11 | 114 | 60 | 174 |
| | (24.3) | (25.0) | (23.4) | (18.6) | (24.1) | (23.5) | (23.9) |
| Oral | 5 | 4 | 4 | 2 | 9 | 6 | 15 |
| | (1.4) | (2.0) | (3.7) | (3.4) | (1.9) | (2.4) | (2.1) |
| Other | 2 | 1 | 0 | 0 | 2 | 1 | 3 |
| | (0.5) | (0.5) | | | (0.4) | (0.4) | (0.4) |

UDS detected opioids at screening^{f}

| | | | | | | | |
|---|---|---|---|---|---|---|---|
| Opioids overall | 360 | 192 | 105 | 58 | 465 | 250 | 715 |
| | (98.6) | (98.5) | (98.1) | (98.3) | (98.5) | (98.4) | (98.5) |
| Fentanyl^{g} | 333 | 178 | 36 | 21 | 369 | 199 | 568 |
| | (91.2) | (91.3) | (33.6) | (35.6) | (78.2) | (78.3) | (78.2) |
| Morphine | 153 | 81 | 94 | 52 | 247 | 133 | 380 |
| | (41.8) | (41.5) | (87.9) | (88.1) | (52.2) | (52.4) | (52.3) |
| Buprenorphine | 19 | 6 | 6 | 4 | 25 | 10 | 35 |
| | (5.2) | (3.1) | (5.6) | (6.8) | (5.3) | (3.9) | (4.8) |
| Methadone | 15 | 8 | 7 | 2 | 22 | 10 | 32 |
| | (4.1) | (4.1) | (6.5) | (3.4) | (4.7) | (3.9) | (4.4) |
| Oxycodone | 9 | 5 | 9 | 8 | 18 | 13 | 31 |
| | (2.5) | (2.6) | (8.4) | (13.6) | (3.8) | (5.1) | (4.3) |
| Opioid overdose history^{h} | 130 | 68 | 28 | 17 | 158 | 85 | 243 |
| | (35.4) | (34.7) | (26.2) | (28.8) | (33.3) | (33.3) | (33.3) |
| More than one overdose | 67 | 41 | 11 | 11 | 78 | 52 | 130 |
| | (18.3) | (20.9) | (10.3) | (18.6) | (16.5) | (20.4) | (17.8) |
| Medically withdrawn from | 275 | 149 | 79 | 44 | 354 | 193 | 547 |
| opioids previouslyⁱ | (74.9) | (76.0) | (73.8) | (74.6) | (74.7) | (75.7) | (75.0) |
| History of prescription | 188 | 92 | 41 | 29 | 229 | 121 | 350 |
| maintenance treatment | (51.2) | (46.9) | (38.3) | (49.2) | (48.3) | (47.5) | (48.0) |
| with buprenorphine, | | | | | | | |
| methadone, or | | | | | | | |
| naltrexoneⁱ | | | | | | | |

Current use of other drugs^{j}

| | | | | | | | |
|---|---|---|---|---|---|---|---|
| Amphetamines or | 256 | 130 | 61 | 41 | 317 | 171 | 488 |
| methamphetamine^{k} | (69.8) | (66.3) | (57.0) | (69.5) | (66.9) | (67.1) | (66.9) |
| Cannabinoids | 221 | 121 | 62 | 35 | 283 | 156 | 439 |
| | (60.2) | (61.7) | (57.9) | (59.3) | (59.7) | (61.2) | (60.2) |
| Cocaine | 106 | 60 | 43 | 22 | 149 | 82 | 231 |
| | (28.9) | (30.6) | (40.2) | (37.3) | (31.4) | (32.2) | (31.7) |
| Benzodiazepines | 76 | 47 | 25 | 17 | 101 | 64 | 165 |
| | (20.7) | (24.0) | (23.4) | (28.8) | (21.3) | (25.1) | (22.6) |
| Phencyclidine | 15 | 13 | 4 | 4 | 19 | 17 | 36 |
| | (4.1) | (6.6) | (3.7) | (6.8) | (4.0) | (6.7) | (4.9) |
| Barbiturates | 9 | 6 | 5 | 6 | 14 | 12 | 26 |
| | (2.5) | (3.1) | (4.7) | (10.2) | (3.0) | (4.7) | (3.6) |

^{a}= shown for the study full analysis set, defined as participants who were randomized to the open-label study treatment, received transmucosal buprenorphine, and did not demonstrate allergic or hypersensitivity reaction (analyzed according to the received induction arm); fentanyl-positive and fentanyl-negative subpopulations are based on the same-day UDS result for fentanyl on the induction day 1 visit of the most recent induction attempt;
^{b}= age at the screening visit;
^{c}= other races were classified as multiple races by the database;
^{d}= opioid lifetime use is calculated as the year of the informed consent date − the start date year + 1;
^{e}= opioid use in the past 4 weeks was by participant self-report;
^{f}= screening UDS opioid results were incomplete for 3 fentanyl-positive participants (2 in the RI arm and 1 in the SI arm);
^{g}= 34.3% of participants who ultimately had a fentanyl-negative finding on UDS at the most recent induction (i.e., fentanyl-negative subpopulation) were fentanyl positive at screening;
^{h}= opioid overdose that required assistance from others, an emergency department visit, or hospitalization;
ⁱ= based on the number of participants in the treatment group;
^{j}= either reporting use (history) or testing positive via UDS at screening;
^{k}= reporting use or testing positive via UDS for amphetamine or methamphetamine.

Table 2 shows that the demographic and baseline characteristics were generally similar between treatment arms. Mean ages were 40.4 and 40.9 years in the SI and RI arms, respectively. Most participants in each treatment arm fell into the age range of 30 to <45 years (59.6% and 53.2% in the SI and RI arms, respectively) or 45 to <60 years (23.5% and 28.1%, respectively). Males comprised 55.7% of the population overall (SI 52.5% and RI 57.4%). Over three quarters of participants in both arms were white (SI 81.2% and RI 75.5%). The majority of participants (83%) in each treatment arm were not Hispanic or Latino. Screening weight and BMI were also similar across treatment arms. Most participants in each treatment arm were current users of nicotine (SI 83.1% and RI 86.1%) and xanthine/caffeine (86.7% and 88.4%, respectively); 20.4% and 23.8%, respectively, reported being current users of alcohol.

As also shown in Table 2, demographic and baseline characteristics were generally similar across fentanyl positive or negative subpopulations (based on same-day UDS results), although there was a marginally higher percentage of women of childbearing potential in the fentanyl positive subpopulation (SI 70.0% and RI 75.0%) compared with the fentanyl negative subpopulation (54.8% and 60.0%, respectively), possibly as a result of that subpopulation being marginally younger (mean ages 39.5 and 40.0 years, respectively) than participants in the fentanyl negative subpopulation (mean ages 43.3 and 44.2 years, respectively).

### Protocol

As shown in FIG. 2, participants who use opioids via an injection route and/or use fentanyl/high doses of opioids were randomly assigned in a 2:1 ratio to the RI arm or SI arm for the induction phase, which was conducted in an open-label setting. Due to the potential for fentanyl use to impact the response to TM BUP, randomization was stratified according to the same-day UDS result for fentanyl (negative or positive).

The RI arm was designed to initiate BUP-XR treatment following a single dose of TM BUP on the day of induction. The participants in the RI arm received 4 mg TM BUP on day 1. After approximately one hour, participants were assessed for COWS and tolerability, and those participants meeting eligibility requirements then received 300 mg BUP-XR (injection 1) at least 1 hour after administration of TM BUP. If necessary, exacerbated withdrawal symptoms were treated with additional TM BUP in increments of 2-4 mg up to 12 mg total TM BUP, inclusive of the initiating dose.

The SI arm initiated the participants onto BUP-XR after giving a TM BUP-containing product for a minimum of 7 days. The participants in the SI arm received injection 1 on day 1 at the conclusion of the administration of TM BUP for a minimum of 7 days. The participants in both the RI and SI arms received another 300 mg BUP-XR (injection 2) on day 8 (up to +4) (i.e. 7 to 11 days after injection 1). After injection 2, BUP-XR was administered monthly using a maintenance dose of 100 mg or 300 mg BUP-XR for up to a total of 8 maintenance injections.

Following injection 2, participants eligible to continue treatment in the maintenance phase were randomized prior to the monthly maintenance dosing of 100 mg or 300 mg BUP-XR in a 1:1 ratio. The monthly maintenance dose of either 300 mg or 100 mg of BUP-XR was administered to the participants in a double-blind manner by subcutaneous injection starting on day 36 (−2/+4) (injection 3), continuing monthly up to 37 weeks from day 1 (injections 4-10).

### Interim Analysis: Sub-Study

A randomized sub-study, nested within the open-label phase of the study, evaluated treatment retention with SI or RI induction to BUP-XR treatment in treatment-seeking participants who frequently injected opioids or used fentanyl/high doses of opioids. Table 3 below shows demographics and baseline characteristics for the sub-study participants.

TABLE 3
Sub-Study Participants

| | Fentanyl POS Subpopulation | | | | | | Fentanyl NEG Subpopulation | | | | | | Total | | | | | |
|---|---|---|---|---|---|---|---|---|---|---|---|---|---|---|---|---|---|---|
| | (N = 96) | | | | | | (N = 41) | | | | | | (N = 137) | | | | | |
| | SI | | RI | | Total | | SI | | RI | | Total | | SI | | RI | | Total | |
| | (N = 35) | | (N = 61) | | (N = 96) | | (N = 13) | | (N = 28) | | (N = 41) | | (N = 48) | | (N = 89) | | (N = 137) | |
| Age, years, Mean (SD) | 42 | (12) | 44 | (11) | 44 | (11) | 44 | (14) | 45 | (11) | 45 | (12) | 43 | (12) | 44 | (11) | 44 | (11) |
| Sex, Male, n (%) | 13 | (37) | 42 | (69) | 55 | (57) | 9 | (69) | 19 | (68) | 28 | (68) | 22 | (46) | 61 | (69) | 83 | (61) |
| Race, n (%) | | | | | | | | | | | | | | | | | | |
| Black or African American | 10 | (29) | 18 | (30) | 28 | (29) | 4 | (31) | 13 | (46) | 17 | (42) | 14 | (29) | 31 | (35) | 45 | (33) |
| White | 24 | (69) | 42 | (69) | 66 | (69) | 9 | (69) | 15 | (54) | 24 | (59) | 33 | (69) | 57 | (64) | 90 | (66) |
| Other | 1 | (3) | 1 | (2) | 2 | (2) | 0 | | 0 | | 0 | | 1 | (2) | 1 | (1) | 2 | (2) |
| Opioid Lifetime Use, years | 13 | (10) | 18 | (11) | 16 | (11) | 18 | (13) | 16 | (8) | 17 | (10) | 15 | (11) | 17 | (10) | 17 | (10) |
| Opioid Use Last 4 Weeks, days | 27 | (2) | 28 | (0) | 28 | (1) | 28 | (0) | 27 | (2) | 28 | (2) | 28 | (2) | 28 | (1) | 28 | (2) |
| Main Route Last 4 Weeks, n (%) | | | | | | | | | | | | | | | | | | |
| Injection | 11 | (31) | 15 | (25) | 26 | (27) | 9 | (69) | 14 | (50) | 23 | (56) | 20 | (42) | 29 | (33) | 49 | (36) |
| Smoking | 10 | (29) | 22 | (36) | 32 | (33) | 1 | (8) | 7 | (25) | 8 | (20) | 11 | (23) | 29 | (33) | 40 | (29) |
| Oral | 1 | (3) | 1 | (2) | 2 | (2) | 1 | (8) | 3 | (11) | 4 | (10) | 2 | (4) | 4 | (5) | 6 | (4) |
| Snorting | 13 | (37) | 23 | (28) | 36 | (38) | 2 | (15) | 4 | (14) | 6 | (15) | 15 | (31) | 27 | (30) | 42 | (31) |

Among the 137 sub-study participants, fentanyl positive (96) and fentanyl negative (41) subpopulations were categorized. Each fentanyl positive or negative participant was randomly assigned in a 2:1 ratio to RI or SI. As Table 3 shows, 13 male participants (37% of total) of the fentanyl positive subpopulation were assigned to SI, and 42 male participants (69% of total) of the fentanyl positive subpopulation were assigned to RI. Further, 9 male participants (69% of total) of the fentanyl negative subpopulation were assigned to SI, and 19 male participants (68% of total) of the fentanyl negative subpopulation were assigned to RI. Randomization was stratified by the same-day UDS fentanyl result.

### Treatment Retention

In the sub-study, the primary endpoint retention rate difference at injection 2 was estimated by Bayesian approach. The posterior probability was evaluated for non-inferiority (NI) of RI to SI using a 10% NI margin against 96% critical value for 1-sided Type I error<10%. Table 4 below presents the results.

TABLE 4
Treatment Retention at Injection 2

| | Fentanyl POS Subpopulation | | Fentanyl NEG Subpopulation | | Overall | |
|---|---|---|---|---|---|---|
| | SI | RI | SI | RI | SI | RI |
| Category | (N = 34) | (N = 60) | (N = 13) | (N = 25) | (N = 47) | (N = 85) |
| Proportion of participants who | 52.9% | 65.0% | 76.9% | 72.0% | 59.6% | 67.1% |
| received Injection 2 (n/N) | (18/34) | (39/60) | (10/13) | (18/25) | (28/47) | (57/85) |
| Difference on retention rate at | | 11.6% | | −2.8% | | 7.5% |
| Injection 2 - (RI − SI) (95% HPD) | | (−8.2, 31.7) | | (−30.1, 24.8) | | (−8.7, 24.5) |
| Probability of RI non-inferior to SI, | | 98.2% | | 68.5% | | 98.2% |
| (RI − SI) > −10% | | | | | | |
| Probability of RI superior to SI | | 87.0% | | 40.8% | | 80.9% |
| (RI − SI) > 0 | | | | | | |

HPD = highest posterior density region; RI = rapid induction; SD = standard deviation; SI = standard of care.

Table 4 shows that the proportion (percentage) of participants who received injection 2 was 52.9% in the SI arm and 65.0% in the RI arm of the fentanyl positive subpopulation, while the proportion was 76.9% in the SI arm and 72.0% in the RI arm in fentanyl negative population. The proportion was 59.6% in the SI arm and 67.1% in RI overall. Table 4 also shows that probability of RI being non-inferior to SI (RI−SI>−10%) was 98.2% in fentanyl positive population, 68.5% in fentanyl negative population, and 98.2% overall. Finally, Table 4 shows that the probability of RI being superior to SI (RI−SI>0) was 87.0% in the fentanyl positive population, 40.8% in the fentanyl negative population, and 80.9% overall.

The observed treatment retention in the RI arm is numerically higher, compared to SI, in both the overall population and among participants who were fentanyl positive on the day of induction. Further, the results indicate that RI is non-inferior to SI since the probability of RI non-inferiority to SI is >98%.

### Precipitated Opioid Withdrawal (POW)

The sub-study evaluated opioid withdrawal symptoms and precipitated opioid withdrawal (POW) symptoms of participants up to injection 2. The results are shown in Table 5.

TABLE 5
Precipitated Opioid Withdrawal up to Injection 2

| | n (%) | | | | | | | |
|---|---|---|---|---|---|---|---|---|
| | Fentanyl Positive (N = 96) | | Fentanyl Negative (N = 41) | | Overall (N = 137) | | | |
| | Category (Participants with) | | | | | | | |
| | SI | RI | SI | RI | SI | | RI | Total |
| | (N = 35) | (N = 61) | (N = 13) | (N = 28) | (N = 48) | | (N = 89) | (N = 137) |
| TEAE reported as opioid | 9 (25.7) | 15 (24.6) | 1 (7.7) | 2 (7.1) | 10 | (20.8) | 17 (19.1) | 27 (19.7) |
| withdrawal symptom | | | | | | | | |
| TEAE reported as precipitated | 8 (22.9) | 13 (21.3) | 0 (0.0) | 2 (7.1) | 8 | (16.7) | 15 (16.9) | 23 (16.8) |
| opioid withdrawal symptom | | | | | | | | |

As seen in Table 5, the number (percentage) of participants who have TEAE reported as opioid withdrawal symptoms was 9 out of 35 participants (25.7%) in the SI arm, and 15 out of 61 participants (24.6%) in the RI arm of the fentanyl positive subpopulation, and 1 out of 13 participants (7.7%) in the SI arm compared to 2 out of 28 participants (7.1%) in the RI arm of the fentanyl negative subpopulation. Overall, 10 out of 48 participants (20.8%) for the SI arm, 17 out of 89 participants (19.1%) for the RI arm, and 27 out of 137 (19.7%) in total had opioid withdrawal symptoms.

Table 5 also shows that the number (percentage) of participants who have TEAE reported as POW symptoms was 8 out of 35 participants (22.9%) in the SI arm and 13 out of 61 participants (21.3%) in the RI arm of the fentanyl positive subpopulation, 0 out of 13 participants (0%) in the SI arm and 2 out of 28 participants (7.1%) in the RI arm of the fentanyl negative subpopulation, and 8 out of 48 participants (16.7%) for the SI arm overall, 15 out of 89 participants (16.9%) for the RI arm overall, and 23 out of 137 (16.8%) in total.

As can be seen from Table 5, more participants who reported POW were fentanyl positive (21/23); thus, POW appears to be a fentanyl-related issue. Most participants reported POW symptoms after TM BUP in RI: n=10 and SI: n=7. All participants who used concomitant medications were in the fentanyl positive subpopulation. The most common medications were clonidine, ondansetron, loperamide, and trazodone. A higher percentage of SI participants (100%) used concomitant medications to treat withdrawal symptoms compared to RI (33.3%) on the first day of induction.

Finally, retention of participants after POW was higher for RI than SI: RI 80.0% vs. SI 37.5% at injection 1; RI 53.3% vs. SI 37.5% at injection 2; and RI 53.3% vs. SI 37.5% at injection 3.

It can be concluded that the incidence of POW is similar between RI and SI, which means that RI does not increase POW. Thus, it can be said that precipitated opioid withdrawal was not more likely for RI. Also, when POW occurred in RI, events were not more serious, severe, or long-lasting, and did not result in an increase in discontinuation compared to SI.

### Clinical Opiate Withdrawal Score (COWS)

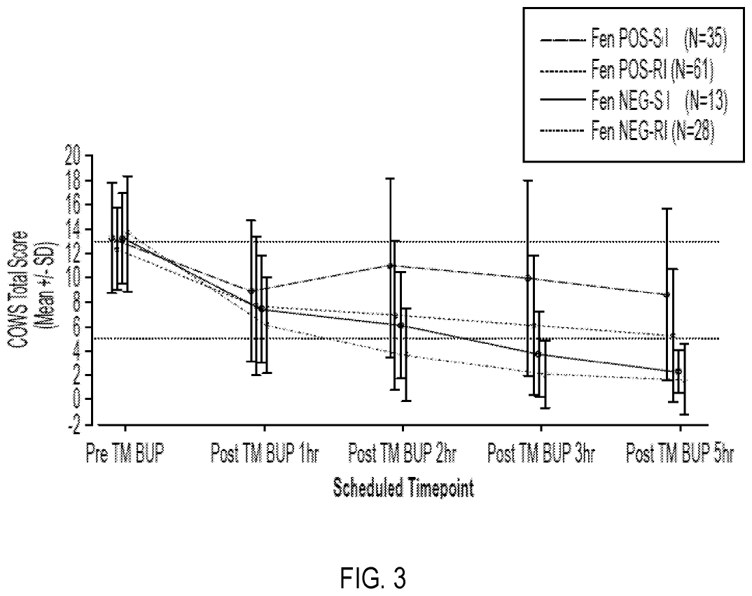

Clinical Opiate Withdrawal Scores (COWS) were evaluated on induction day pre-dose, and at 1, 2, 3, and 5 hours post-TM BUP. The results are shown in FIG. 3. As FIG. 3 shows, the response of opioid withdrawal symptoms was comparable for SI and RI overall. COWS scores tended to be lower in the RI arm compared to the SI arm during induction. FIG. 3 also shows that fentanyl positive participants tended to have higher COWS scores during induction.

### Treatment-Emergent Adverse Events (TEAEs)

Treatment-emergent adverse events (TEAEs) were evaluated up to injection 2 in the sub-study. The results are shown in Table 6.

TABLE 6
TEAE Profile Up to Injection 2

| | n (%) | | | | | | | | | | | |
|---|---|---|---|---|---|---|---|---|---|---|---|---|
| | Fentanyl Positive | | | | Fentanyl Negative | | | | Overall | | | |
| | (N = 96) | | | | (N = 41) | | | | (N = 137) | | | |
| | Category (Participants with) | | | | | | | | | | | |
| | SI | | RI | | SI | | RI | | SI | | RI | Total |
| | (N = 35) | | (N = 61) | | (N = 13) | | (N = 28) | | (N = 48) | | (N = 89) | (N = 137) |
| Any TEAE up to Injection 2 | 14 | (40.0) | 20 | (32.8) | 4 | (30.8) | 6 | (21.4) | 18 | (37.5) | 26 (29.2) | 44 (32.1) |
| TM BUP-related TEAEs | 7 | (20.0) | 8 | (13.1) | 0 | (0.0) | 2 | (7.1) | 7 | (14.6) | 10 (11.2) | 17 (12.4) |
| RBP-6000 related TEAEs | 6 | (17.1) | 10 | (16.4) | 3 | (23.1) | 2 | (7.1) | 9 | (18.8) | 12 (13.5) | 21 (15.3) |
| Serious TEAEs | 0 | (0.0) | 1 | (1.6) | 0 | (0.0) | 0 | (0.0) | 0 | (0.0) | 1 (1.1) | 1 (0.7) |
| TEAEs leading to | 0 | (0.0) | 2 | (3.3) | 0 | (0.0) | 2 | (7.1) | 0 | (0.0) | 4 (4.5) | 4 (2.9) |
| discontinuation | | | | | | | | | | | | |

As seen in Table 6, 35 participants of the SI arm and 61 participants of the RI arm among fentanyl positive subpopulation, and 13 participants of the SI arm and 28 participants of the RI arm among fentanyl negative population, are categorized. Table 6 shows that any TEAE up to injection 2 was 14 out of 35 participants in the SI arm compared to 20 out of 61 participants in the RI arm for the fentanyl positive population, and 4 out of 13 participants in the SI arm compared to 6 out of 28 participants in the RI arm in the fentanyl negative population. There were 18 out of 48 participants in the SI arm overall, 26 of 89 participants in the RI arm overall, and 44 out of 137 in total, with TEAEs. The number of participants who experienced received TM BUP-related TEAEs was 7 out of 35 participants in the SI arm, 8 out of 61 participants in the RI arm in the fentanyl positive population, and 0 out of 13 participants in the SI arm compared to 2 out of 28 participants in the RI arm in the fentanyl negative population, 7 out of 48 participants in the SI arm overall, 10 of 89 in the RI arm overall, and 17 out of 137 in total.

The number of participants who experienced BUP-XR related TEAEs was 6 out of 35 participants in the SI arm compared to 10 out of 61 participants in the RI arm in the fentanyl positive population and 3 out of 13 participants in the SI arm compared to 2 out of 28 in the RI arm in the fentanyl negative population, with 9 out of 48 participants in SI overall, 12 of 89 in RI overall, and 21 out of 137 in total. The number of participants who experienced serious TEAEs was 0 out of 35 participants in the SI arm compared to 1 out of 61 participants in the RI arm in the fentanyl positive population and 0 out of 13 participants in the SI arm compared to 0 out of 28 participants in the RI arm in the fentanyl negative population, with 0 out of 48 participants in SI overall, 1 of 89 in RI overall, and 1 out of 137 in total. The number of participants experiencing TEAEs leading to discontinuation was 0 out of 35 participants in the SI arm and 2 out of 61 participants in RI arm in the fentanyl positive population, and 0 out of 13 participants in the SI arm and 2 out of 28 participants in the RI arm in the fentanyl negative population, with 0 out of 48 participants in SI overall, 4 of 89 in RI overall, and 4 out of 137 in total.

In SI, up to injection 3, it can be said that administration of BUP-XR injection 2 as soon as 1 week (rather than 4 weeks) after injection 1 (day 1 for both RI and SI) was well tolerated. There were some adverse events from this study of RI and SI. For example, there was a small decline in the number of participants reporting TEAEs before 300 mg of BUP-XR compared to after 300 mg of BUP-XR. However, there was no individual adverse event observed in 5% of participants between injections 2 and 3.

This sub-study demonstrates that, compared to standard induction, rapid induction had better outcomes for both treatment retention and precipitated opioid withdrawal in this high-risk population. Administration of injection 2 was safe and well tolerated. The shorter time necessary for RI may increase feasibility of BUP-XR treatment, particularly in individuals who use fentanyl.

### Final Analysis

Once the study was complete, final analysis of the data shows that RI treatment is surprisingly superior to SI treatment. The primary end point (treatment retention at injection 2) was estimated by Bayesian β-binomial model using non-informative uniform prior within individual induction arms. The posterior distribution of retention rate difference between the 2 arms was used to calculate the posterior probability of non-inferiority of RI to SI using a 10% noninferiority margin or a favorable trend of RI over SI. The criterion for non-inferiority was defined as having a 96% or greater posterior probability (the critical value) that the retention rate difference (RI−SI) was greater than ~10%. If RI was noninferior to SI, the posterior possibility of retention rate difference (RI−SI) greater than 0 was estimated to evaluate superiority using the same critical value. The 95% credible interval (CrI) was also estimated via highest posterior density region. Retention at injection 3 was estimated in the same way.

Study retention from the first transmucosal buprenorphine dose was plotted by induction arm using Kaplan-Meier curves within the fentanyl UDS subpopulations. The time from the first transmucosal buprenorphine dose to study discontinuation was derived as the date of the last scheduled visit observed minus the date of the first dose of transmucosal buprenorphine+1. Study completion was censored administratively at the time of injection 3. Retention rate at the end of 5 weeks (35 days) was estimated using the Kaplan-Meier product limit method for each induction arm within the fentanyl UDS subpopulation. No data for treatment or study discontinuation were missing. For other end points, an as observed approach without imputation was used for analysis. Statistical analyses were conducted using SAS version 9.4 or SAS EG version 7.1.

A total of 777 of all 785 randomized participants (99.0%) received transmucosal buprenorphine (8 discontinued before dosing). Forty-eight participants (5.6%) received inductions deviating from the study protocol. The remaining 729 participants (92.9%) were included in the safety full analysis set (SFAS) (474 in the RI arm and 255 in the SI arm), of whom 6 did not meet study eligibility criteria (protocol violations) and were discontinued after randomization. The remaining 723 participants (92.1%) were evaluable for treatment retention or study discontinuation (evaluable population).

Table 7A below shows disposition of all randomized participants, and Table 7B shows disposition of the SFAS through injection 3.

TABLE 7A
Disposition of Randomized Participants

Overall (n = 785)

| | SI (n = 267) | RI (n = 518) |
|---|---|---|
| Induction (TM BUP) (%) | 264 (98.9%) | 513 (99.0%) |
| Week 1; Injection 1 (%) | 159 (59.6%) | 444 (85.7%) |
| Week 2; Injection 2 (%) | 145 (54.3%) | 339 (65.4%) |
| Week 6; Injection 3 (%) | 137 (51.3%) | 298 (57.5%) |

TABLE 7B
Disposition of SFAS Participants

| | Fentanyl Positive | | Fentanyl Negative | | Overall | |
|---|---|---|---|---|---|---|
| | N = 563 | | N = 166 | | n = 729* | |
| | Category (n) | | | | | |
| | SI | RI | SI | RI | SI | RI |
| | (N = 196) | (N = 367) | (N = 59) | (N = 107) | (N = 255) | (N = 474) |
| Induction (TM BUP) (%) | 100.0% | 100.0% | 100.0% | 100.0% | 100.0% | 100.0% |
| Injection 1 (%) | 52.0% | 84.5% | 83.1% | 92.5% | 151 (59.2%) | 409 (86.3%) |
| Injection 2 (%) | 47.4% | 62.4% | 76.3% | 79.4% | 138 (54.1%) | 314 (66.2%) |
| Injection 3 (%) | 44.4% | 56.4% | 71.2% | 68.1% | 129 (50.6%) | 278 (58.6%) |

*Includes the 6 non-eligible participants who were discontinued.

### Treatment Retention

With respect to participant retention through injection 2 (participants receiving injection 2 who received at least 1 dose of TM BUP for induction and did not demonstrate idiosyncratic response to the first dose of TM BUP), the RI arm showed superiority to the SI arm. The retention results are summarized in Table 8.

TABLE 8
Treatment Retention at Injection 2

| | Fentanyl positive | | Fentanyl negative | | Total | |
|---|---|---|---|---|---|---|
| | (n = 560) | | (n = 163) | | (N = 723)^{a} | |
| | RI | SI | RI | SI | RI | SI |
| | (n = 366) | (n = 194) | (n = 104) | (n = 59) | (n = 470) | (n = 253) |
| Proportion of | 230 | 93 | 82 | 45 | 312 | 138 |
| participants who | (62.8) | (47.9) | (78.8) | (76.3) | (66.4) | (54.5) |
| received injection 2, | | | | | | |
| No. (%)^{b,c} | | | | | | |
| Difference in | 14.8 | | 3.0 | | 11.8 | |
| retention rate at | (6.5 to | | (−10.5 to | | (4.3 to | |
| injection 2, RI − SI | 23.7) | | 16.1) | | 19.0) | |
| (95% Crl)^{d} | | | | | | |
| Posterior probability | 100.0 | | 97.4 | | 100.0 | |
| of RI noninferior to SI | | | | | | |
| (i.e., RI − SI > −10%)^{e} | | | | | | |
| Posterior probability | 100.0 | | 66.3 | | 99.9 | |
| of RI superior to SI | | | | | | |
| (i.e., RI − SI > 0)^{f} | | | | | | |
| Proportion of | 207 | 87 | 70 | 42 | 277 | 129 |
| participants who | (56.6) | (44.8) | (67.3) | (71.2) | (58.9) | (51.0) |
| received injection 3, | | | | | | |
| No. (%)^{g} | | | | | | |
| Difference in | 11.6 | | −3.5 | | 7.9 | |
| retention rate at | (3.1 to | | (−18.2 to | | (0.2 to | |
| injection 3, RI − SI | 20.2) | | 10.6) | | 15.3) | |
| (95% Crl)^{d,g} | | | | | | |
| Posterior probability | 99.6 | | 31.4 | | 97.9 | |
| of RI superior to SI | | | | | | |
| (i.e., RI − SI > 0)^{f} | | | | | | |

^{a}= shown for the evaluable population, defined as randomized participants who were randomized to the open-label study treatment, met the inclusion and exclusion criteria, received transmucosal buprenorphine for induction, and did not demonstrate an allergic or hypersensitivity reaction to transmucosal buprenorphine;
^{b}= retention rate at injection 2 is defined as the proportion of participants receiving injection 2 at the week 2 visit among those in the evaluable population for treatment retention or discontinuation;
^{c}= numerator of the proportion is the number of participants who received injection 2 at the week 2 (nominal) visit or missed injection 2 at week 2 (nominal) visit but received injection 3 at week 6 day 36 visit (denominator is the number of participants in the evaluable population);
^{d}= estimated by Bayesian β-binomial model using noninformative uniform prior (i.e., β[1, 1]), within individual induction arms;
^{e}= noninferiority of RI to SI is declared if the posterior probability is at 96% or greater;
^{f}= if the noninferiority of RI to SI is declared, the posterior possibility of retention rate difference (RI − SI) greater than 0 will be estimated to evaluate the superiority of RI over SI induction;
^{g}= retention at injection 3 is defined as the proportion of participants receiving injection 3 at the week 6 visit among those in the evaluable population.

As Table 8 shows, RI treatment was superior to SI treatment with respect to participant retention through injection 2. The proportion of participants who received injection 2 was higher in the RI arm (66.4%) compared with the SI arm (54.5%), and the estimated treatment retention rates at injection 2 were 66.3% and 54.5%, respectively. The difference (95% highest posterior density (“HPD”)) in the retention rate at injection 2 was 11.82% (4.34%, 19.03%) in favor of RI treatment.

The results for the fentanyl positive subpopulation were supportive of the overall results (RI=62.8% and SI=47.9%; estimated treatment retention rates at injection 2=62.7% and 47.9%, respectively; difference [95% HPD]=14.84% [6.51%, 23.72%]), while in the fentanyl negative subpopulation, the treatment retention rates for the 2 treatment arms were numerically comparable (SI=75.3% and RI=78.3%). When the primary endpoint was analysed as “randomized,” as well as in additional sensitivity analyses, the results were very similar to those in the primary analysis, demonstrating the robust nature of these findings.

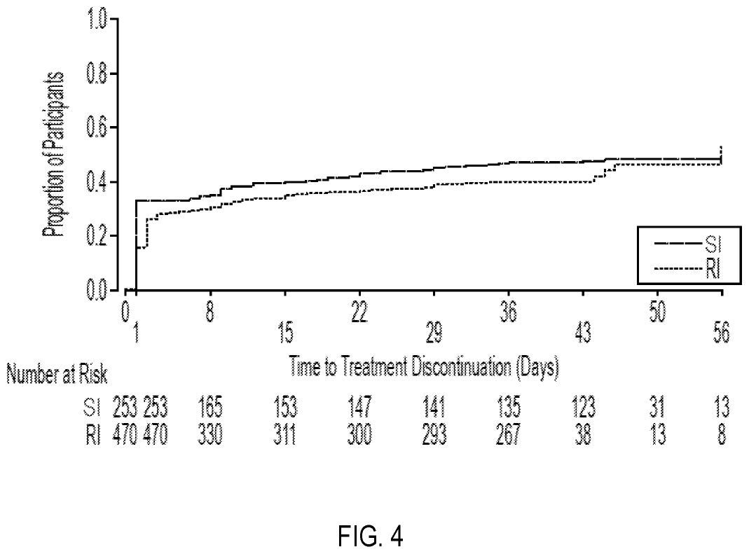

Additionally, as shown in FIG. 4, the time to treatment discontinuation among those in the evaluable population for treatment retention/discontinuation favored the RI treatment arm compared with the SI treatment arm, with hazard ratio=0.82 for RI/SI, 95% CI (0.65, 1.03), accounting for the fentanyl UDS subpopulation. Even though the SI arm included an extra week of TM BUP induction compared with the RI arm, the retention rate at 5 weeks post-injection 1 was higher in the RI arm compared with the SI arm (60.2% and 53.4%, respectively).

### Precipitated Opioid Withdrawal (POW) and Clinical Opioid Withdrawal Score (COWS)

The evaluation of POW was an exploratory endpoint. Per protocol, investigators were instructed to evaluate whether POW occurred and, if so, to document any associated adverse events. An additional analysis was conducted in which POW was defined as an increase of at least 6 points in the COWS total score, measured within 5 hours following TM BUP administration. Thus, the evaluation focused on precipitated withdrawal based on both the investigator assessment and the COWS score increase of at least 6 points. Table 9 below presents a comparison of POW events between the RI arm and the SI arm.

TABLE 9
Comparison of POW Events

| | n (%) | | | | | | | | | |
|---|---|---|---|---|---|---|---|---|---|---|
| | Fentanyl Positive | | | | Fentanyl negative | | Overall | | | |
| | N = 563 | | | | N = 166 | | N = 729 | | | |
| | POW Category (Participants with) | | | | | | | | | |
| | SI | | RI | | SI | RI | SI | | RI | |
| | (N = 196) | | (N = 367) | | (N = 59) | (N = 107) | (N = 255) | | (N = 474) | |
| Investigator Assessed | 44 | (22.4) | 107 | (29.2) | 2 (3.4) | 7 (6.5) | 46 | (18.0) | 114 | (24.1) |
| RI-SoC (95% CI) | | | | | | | 6.01% (−0.30%, 11.92%) | | | |
| COWS increase ≥6 | 27 | (13.8) | 51 | (33.9) | 2.(3.4) | 2 (1.9) | 29 | (11.4) | 53 | (11.2) |
| Investigator Assessed AND COWS | 1.6 | (8.2) | 34 | (9.3) | 0 | 1 (0.9) | 16 | (6.3) | 35 | (7.4) |
| increase ≥6 | | | | | | | | | | |

As shown in Table 9, the percentage of participants who experienced POW during induction was similar between treatment arms, whether analyzed based on investigator assessment of POW or on a COWS score increase of at least 6 points. Importantly, retention of participants with investigator-assessed POW was higher for RI than for SI: at injection 2, the difference in retention was 13.72% (95% CI −2.3, 29.8).

Most participants who experienced POW were fentanyl positive at baseline. Most treatment-emergent adverse events (TEAEs) associated with POW were of mild or moderate intensity. Among participants who experienced POW, retention through injection 2 and injection 3 was higher for the RI arm than for the SI arm.

While there is currently no universally accepted definition of precipitated withdrawal, recent publications suggest that an increase in COWS of at least 6 is a reasonable and objective cut point for precipitated withdrawal. Similar percentages of participants in the SI (11.4%) and RI (11.2%) arms had a COWS total score increase from pre-TM BUP of at least 6 on the most recent induction day at any time within the 5 hours on site.

With respect to TEAEs associated with precipitated withdrawal, similar percentages of participants in the SI and RI arms reported TEAEs associated with precipitated withdrawal post TM BUP (16.1% and 12.7% respectively) and post BUP-XR in the RI arm (13.9%), while a lower percentage of participants in the SI arm experienced these events (4.0%). These events were more common in the fentanyl positive subpopulation compared with the fentanyl negative subpopulation.

Most participants in the RI arm, even those who experienced events of precipitated withdrawal after TM BUP, received BUP-XR within 1 to 2 hours after the first TM BUP administration, so withdrawal symptoms did not result in delayed BUP-XR injection.

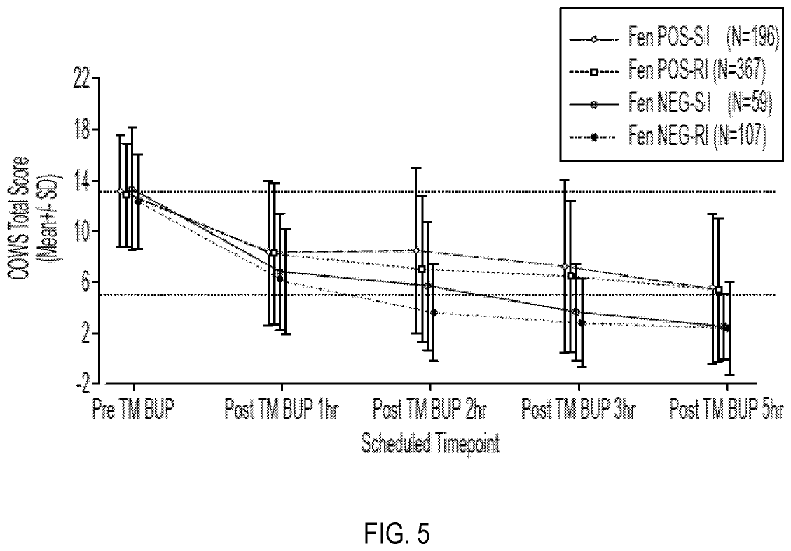

On induction day, the response of opioid withdrawal symptoms, as measured by COWS total scores, was comparable for both the SI and RI arms overall and within the fentanyl subpopulation, although symptoms were more pronounced in participants in the fentanyl positive subpopulation. This is shown in FIG. 5. Participants in the RI arm received TM BUP followed by BUP-XR at 1 hour, while those in the SI arm received only TM BUP. In most individuals, the COWS total score peaked at 2 to 3 hours post-TM BUP, which corresponds to 1 to 2 hours post-injection in the RI group. These findings suggest that observation for 3 hours post-TM BUP (i.e., 2 hours post-BUP-XR) could be recommended for RI.

In a comparison of treatment arms, the percentage of participants experiencing POW was similar for both the RI and SI arms, with no significant differences in investigator-assessed or changes in COWS events. Most POW events were rated as mild or moderate in severity, with less than 2% classified as severe, and no participants recorded COWS scores in the severe range (>36). However, a marginally higher percentage of participants in the SI arm exhibited moderately severe scores (25-36) compared to those in the RI arm. Regarding the use of concomitant medications to manage withdrawal symptoms, 80.5% of the SI arm and 72% of the RI arm utilized such treatments, with clonidine, ondansetron, and trazodone being the most commonly prescribed.

### Treatment-Emergent Adverse Events (TEAEs)

Overall TEAE results of the study are shown in Table 10.

TABLE 10
Overall Treatment-Emergent Adverse Events to Injection 2

| | n (%) | | | | | | | | | | | |
|---|---|---|---|---|---|---|---|---|---|---|---|---|
| | Fentanyl Positive | | | | Fentanyl Negative | | | Overall | | | | |
| | N = 563 | | | | N = 166 | | | N = 729 | | | | |
| | Category (Participants with) | | | | | | | | | | | |
| | SI | | RI | | SI | | RI | SI | RI | | Total | |
| | (N = 196) | | (N = 367) | | (N = 59) | | (N = 107) | (N = 255) | (N = 474) | | (N = 729) | |
| Any TEAE up to Injection 2 | 72 | (36.7) | 164 | (44.7) | 21 | (35.6) | 38 (35.5) | 93 (36.5) | 202 | (42.6) | 295 | (40.5) |
| Severe TEAEs | 6 | (3.1) | 15 | (4.1) | 0 | | 2 (1.9) | 6 (2.4) | 17 | (3.6) | 23 | (3.2) |
| Serious TEAEs | 1 | (0.5) | 5 | (1.4) | 0 | | 0 | 1 (0.4) | 5 | (1.1) | 6 | (0.8) |
| TEAEs potentially related to | 1 | (0.5) | 1 | (0.3) | 0 | | 0 | 1 (0.4) | 1 | (0.2) | 2 | (0.3) |
| Hepatic disorders | | | | | | | | | | | | |
| TEAE reported as Opioid | 55 | (28.1) | 137 | (37.3) | 9 | (15.3) | 12 (11.2) | 64 (25.1) | 149 | (31.4) | 213 | (29.2) |
| withdrawal symptom | | | | | | | | | | | | |
| TM BUP-related TEAEs | 45 | (23.0) | 68 | (18.5) | 9 | (15.3) | 7 (6.5) | 54 (21.2) | 75 | (15.8) | 129 | (17.7) |
| TEAEs leading to | 1 | (0.5) | 2 | (0.5) | 0 | | 0 | 1 (0.4) | 2 | (0.4) | 3 | (0.4) |
| discontinuation from TM BUP | | | | | | | | | | | | |

Table 10 shows the overall and TM BUP TEAE profile up to injection 2 was comparable for the RI and SI arms. The percentage of participants in the SFAS with TEAEs up to injection 2 was 36.5% in the SI arm compared with 42.6% in the RI arm (% difference RI−SI (95% CI)=6.1 (−1.3, 13.4); among participants in the SFAS who actually received BUP-XR, these values were 40.4% and 40.8% (0.4 (−8.8, 9.4)). The percentages of participants in the SFAS with TEAEs up to injection 2 were not different between treatment arms in the fentanyl UDS subpopulations: 36.7% in the SI arm and 44.7% the RI arm in the fentanyl positive subpopulation (% difference RI−SI (95% CI)=8.0 (−0.6, 16.2)) and 35.6% and 35.5%, respectively, in the fentanyl negative subpopulation (−0.1 (−15.6, 14.6)).

Most TEAEs up to injection 2 were of mild or moderate intensity. Severe TEAEs were reported in low and similar percentages of participants in the SI and RI arms (2.4% and 3.6%, respectively; % difference RI−SI [95% Cl]=1.2 [−1.7, 3.7]).

Serious TEAEs up to injection 2 were reported for 1 participant (0.4%) in the SI arm and 5 participants (1.1%) in the RI arm, all in participants in the fentanyl positive subpopulation (% difference RI−SI [95% CI]=0.7 [−1.2, 2.1]). None of these was fatal or met the liver function test criterion of ALT or AST values >3×ULN (upper limit of normal) and bilirubin >2×ULN. Two participants, 1 in each treatment arm, had TEAEs potentially related to hepatic disorders (per customized Medical Dictionary for Regulatory Activities query definition).

Three participants discontinued TM BUP as a result of TEAEs up to injection 2 (SI=1 participant [0.4%] and RI=2 participants [0.4%]; % difference RI−SI [95% CI]=0.0 [−1.8, 1.2]). Among participants in the SFAS who actually received BUP-XR, 5 participants (all RI, 1.2% among those in the RI who received BUP-XR) discontinued BUP-XR as the result of a TEAE up to injection 2 (0.3 [−1.8, 1.8]).

There was a notable decline in TEAEs between injections 2 and 3 compared to the rates observed up to injection 2. Specifically, the percentage of participants experiencing TEAEs between injections 2 and 3 was lower for both the SI arm (21.0% vs. 36.5%) and the RI arm (27.4% vs. 42.6%). The TEAE profile remained consistent across both SI and RI arms. Additionally, two participants experienced serious adverse events (SAEs), one of which was fatal, and two participants had TEAEs that led to discontinuation from BUP-XR; however, none of these events were related to the medication.

### CONCLUSIONS

A total of 560 participants received extended-release buprenorphine injection 1 (409 (86.3%) and 151 (59.2%) in the RI and SI treatment arms, respectively), 452 participants received extended-release buprenorphine injection 2 (314 (66.2%) and 138 (54.1%), respectively), and 407 participants received injection 3 (278 (58.6%) and 129 (50.6%), respectively), thereby completing the study. A total of 408 of 474 participants (86.1%) in the RI arm and 219 of 255 participants (85.9%) in the SI arm had a single transmucosal buprenorphine induction attempt, and 56 of 474 (11.8%) in the RI arm and 31 of 255 participants (12.2%) in the SI arm had 2 attempts. Among RI participants who received injection 1, 415 of the 474 total patients (87.6%), 317 of 367 fentanyl-positive patients (86.4%), and 98 of 107 fentanyl-negative patients (91.6%) did not require the use of additional transmucosal buprenorphine on day 1; none had a transmucosal buprenorphine dose greater than 8 mg after extended-release buprenorphine. Among the 154 SI participants, all but 3 reported induction periods of 7 days or longer. On the first transmucosal buprenorphine induction day (during the 5 hours on-site), in the SI arm, 111 participants received a total transmucosal buprenorphine dose of 4 mg or less, 120 participants received 6 to 12 mg, and 24 participants received more than 12 mg.

The difference in the retention rate between RI and SI at injection 2 was 11.8% (95% CrI, 4.3%-19.0%; posterior probability >0 was 99.9%). There were similar findings in the fentanyl-positive subpopulation—a difficult-to-treat population—for which the retention rate difference was 14.8% (95% CrI, 6.5%-23.7%; posterior probability >0 was >99.9%). Among the fentanyl-negative subpopulation, both treatment arms had numerically comparable retention (difference, 3.0%; 95% CrI, −10.5% to 16.1%). A higher proportion of participants in the RI treatment arm compared to the SI treatment arm received injection 3 (posterior probability for RI superior to SI was 97.9%), demonstrating that increased retention rates were achieved with RI. Despite the extra week of transmucosal buprenorphine induction with SI compared to RI, the study retention rate at 5 weeks after transmucosal buprenorphine was higher in the RI arm than in the SI arm (60.2% vs 53.4%). The time to study discontinuation accounting for fentanyl UDS subpopulation favored the RI treatment arm (RI and SI hazard ratio, 0.82; 95% CrI, 0.65-1.03). A total of 565 participants (77.5%) were fentanyl positive by UDS at the induction visit. Induction failure was much higher in the fentanyl-positive SI subpopulation (94 of 196 (48.0%)) compared with the fentanyl-negative subpopulation (10 of 59 (16.9%)). After injection 1 and before injection 2 at week 2, 4 of 49 (8.2%) of the fentanyl-negative and 9 of 102 (8.8%) of the fentanyl-positive subpopulations discontinued. Before injection 3 at week 6, 7 of 49 (14.3%) and 15 of 102 (14.7%) of the fentanyl-negative and fentanyl-positive subpopulations, respectively, discontinued.

The results of this study surprisingly demonstrate that RI treatment was superior to SI treatment regarding participant retention through injection 2. The RI regimen was found to be safe and well tolerated, with POW events not being more likely to occur or be more severe with the RI regimen than with the SI regimen. Notably, most POW events (151 of 160) occurred in participants who were fentanyl positive at baseline. Among those who experienced precipitated withdrawal, retention rates through injection 2 and injection 3 were higher for the RI group compared to the SI group. Additionally, the administration of BUP-XR injection 2 as soon as 1 week (rather than 4 weeks) after injection 1 was well tolerated under the conditions of this study, and pharmacokinetic data support similar plasma exposure to buprenorphine when compared to standard administration at 4 weeks.

These results therefore establish that rapid buprenorphine induction in treatment of OUD increases success in both induction and retention rates, and reduces POW events, even in fentanyl-positive patients. The fact that this protocol achieved a statistically significant improvement (14.8%) in this specific high-risk group was surprising as it was expected that the fentanyl-positive group would have had lower retention and higher rates of precipitated withdrawal. Without being bound by theory, it is believed that the protocol results in target therapeutic buprenorphine plasma concentration levels being achieved sooner than with SI methods, which levels are then successfully maintained with monthly administration of BUP-XR. The RI methods, therefore, can be used to minimize POW and/or increase patient retention in OUD treatment with BUP-XR, increasing the likelihood of successful outcomes.

1. A method of treating opioid use disorder in a human in need thereof, the method comprising:
(a) administering a composition comprising about 4 mg of transmucosal buprenorphine hydrochloride to the human on day 1,
(b) administering about 300 mg of BUP-XR by subcutaneous injection to the human at least 1 hour later.

2. A method of treating opioid use disorder in a human in need thereof, the method comprising:
(a) administering a composition comprising about 4 mg of transmucosal buprenorphine hydrochloride to the human on day 1,
(b) administering about 300 mg of BUP-XR by subcutaneous injection 1 at least 1 hour later,
(c) administering a second dose of 300 mg of BUP-XR to said human by subcutaneous injection 2 as early as day 8 or up to one month after injection 1,
(d) administering 100 mg or 300 mg of BUP-XR monthly to the human by subcutaneous injection 3 starting one month from administration of said injection 2 and continuing monthly as long as needed,
wherein, at least one of the following results is obtained:
(i) completion of steps (a) through (b) has a retention rate superior to the standard of care induction at an endpoint of injection 2;
(ii) completion of steps (a) though (b) does not increase Precipitated Opioid Withdrawal (POW) relative to the standard of care induction;
(iii) for patients experiencing POW, retention at injection 1 is higher after completion of step (a) with RI than for standard of care induction; retention at injection 2 is higher after completion of steps (a) through (b) than for the standard of care induction; and retention at injection 3 is higher after completion of steps (a) through (c) than for standard of care induction;
(iv) the Clinical Opiate Withdrawal Score (COWS) from completion of steps (a) through (b) during RI is lower than for the standard of care induction;
(v) treatment-emergent adverse events (TEAEs) up to the second injection in the steps (a) through (b) are comparable to the TEAEs up to the second injection for the standard of care induction; or
(vi) treatment-emergent adverse events (TEAEs) up to the third injection in the steps (a) through (c) were comparable to the TEAEs up to the third injection for the standard of care induction.
